SEQUENCE LISTING (155 sequences)

SEQ ID NO: 1 - DNA, 2732 bp, Homo sapiens
ctttcgcctcagtctcgagctctcgctggccttcgggtgtacgtgctccgggatcttcagcacccgcggccgccatcgccgtcgcttggcttcttctggactcatctgcgccacttgtccgcttcacactccgccgccatcatggtgaagctcgcgaaggcaggtaaaaatcaaggtgaccccaagaaaatggctcctcctccaaaggaggtagaagaagatagtgaagatgaggaaatgtcagaagatgaagaagatgatagcagtggagaagaggtcgtcatacctcagaagaaaggcaagaaggctgctgcaacctcagcaaagaaggtggtcgtttccccaacaaaaaaggttgcagttgccacaccagccaagaaagcagctgtcactccaggcaaaaaggcagcagcaacacctgccaagaagacagttacaccagccaaagcagttaccacacctggcaagaagggagccacaccaggcaaagcattggtagcaactcctggtaagaagggtgctgccatcccagccaagggggcaaagaatggcaagaatgccaagaaggaagacagtgatgaagaggaggatgatgacagtgaggaggatgaggaggatgacgaggacgaggatgaggatgaagatgaaattgaaccagcagcgatgaaagcagcagctgctgcccctgcctcagaggatgaggacgatgaggatgacgaagatgatgaggatgacgatgacgatgaggaagatgactctgaagaagaagctatggagactacaccagccaaaggaaagaaagctgcaaaagttgttcctgtgaaagccaagaacgtggctgaggatgaagatgaagaagaggatgatgaggacgaggatgacgacgacgacgaagatgatgaagatgatgatgatgaagatgatgaggaggaggaagaagaggaggaggaagagcctgtcaaagaagcacctggaaaacgaaagaaggaaatggccaaacagaaagcagctcctgaagccaagaaacagaaagtggaaggcacagaaccgactacggctttcaatctctttgttggaaacctaaactttaacaaatctgctcctgaattaaaaactggtatcagcgatgtttttgctaaaaatgatcttgctgttgtggatgtcagaattggtatgactaggaaatttggttatgtggattttgaatctgctgaagacctggagaaagcgttggaactcactggtttgaaagtctttggcaatgaaattaaactagagaaaccaaaaggaaaagacagtaagaaagagcgagatgcgagaacacttttggctaaaaatctcccttacaaagtcactcaggatgaattgaaagaagtgtttgaagatgctgcggagatcagattagtcagcaaggatgggaaaagtaaagggattgcttatattgaatttaagacagaagctgatgcagagaaaacctttgaagaaaagcagggaacagagatcgatgggcgatctatttccctgtactatactggagagaaaggtcaaaatcaagactatagaggtggaaagaatagcacttggagtggtgaatcaaaaactctggttttaagcaacctctcctacagtgcaacagaagaaactcttcaggaagtatttgagaaagcaacttttatcaaagtaccccagaaccaaaatggcaaatctaaagggtatgcatttatagagtttgcttcattcgaagacgctaaagaagctttaaattcctgtaataaaagggaaattgagggcagagcaatcaggctggagttgcaaggacccaggggatcacctaatgccagaagccagccatccaaaactctgtttgtcaaaggcctgtctgaggataccactgaagagacattaaaggagtcatttgacggctccgttcgggcaaggatagttactgaccgggaaactgggtcctccaaagggtttggttttgtagacttcaacagtgaggaggatgccaaagctgccaaggaggccatggaagacggtgaaattgatggaaataaagttaccttggactgggccaaacctaagggtgaaggtggcttcgggggtcgtggtggaggcagaggcggctttggaggacgaggtggtggtagaggaggccgaggaggatttggtggcagaggccggggaggctttggagggcgaggaggcttccgaggaggcagaggaggaggaggtgaccacaagccacaaggaaagaagacgaagtttgaatagcttctgtccctctgctttcccttttccatttgaaagaaaggactctggggtttttactgttacctgatcaatgacagagccttctgaggacattccaagacagtatacagtcctgtggtctccttggaaatccgtctagttaacatttcaagggcaataccgtgttggttttgactggatattcatataaactttttaaagagttgagtgatagagctaacccttatctgtaagttttgaatttatattgtttcatcccatgtacaaaaccattttttcctacaaatagtttgggttttgttgttgtttcttttttttgttttgtttttgttttttttttttttgcgttcgtggggttgtaaaagaaaagaaagcagaatgttttatcatggtttttgcttcagcggctttaggacaaattaaaagtcaactctggtgccagaaaaaaaaaaaaaaaa

SEQ ID NO: 2 - DNA, 20 bp, Artificial sequence
  Primer
caggtctccaaggtgaacag

SEQ ID NO: 3 - DNA, 22 bp, Artificial sequence
  Primer
cttagagccaatccttatcccg

SEQ ID NO: 4 - DNA, 28 bp, Artificial sequence
  Probe
tcccttacctacattgttccaacatgcc

SEQ ID NO: 5 - DNA, 21 bp, Artificial sequence
  Primer
gcttggcttcttctggactca

SEQ ID NO: 6 - DNA, 18 bp, Artificial sequence
  Primer
tcgcgagcttcaccatga

SEQ ID NO: 7 - DNA, 24 bp, Artificial sequence
  Probe
cgccacttgtccgcttcacactcc

SEQ ID NO: 8 - DNA, 26 bp, Artificial sequence
  Primer
aatggctaagtgaagatgacaatcat

SEQ ID NO: 9 - DNA, 25 bp, Artificial sequence
  Primer
tgcacatatcattacaccagttcgt

SEQ ID NO: 10 - DNA, 30 bp, Artificial sequence
  Probe
ttgcagcaattcactgtaaagctggaaagg

SEQ ID NO: 11 - DNA, 18 bp, Artificial sequence
  Primer
tcctgcgcggttgttctc

SEQ ID NO: 12 - DNA, 17 bp, Artificial sequence
  Primer
ggcggcacgcacttagg

SEQ ID NO: 13 - DNA, 26 bp, Artificial sequence
  Probe
cagcgttcttttatctccgtccgcct

SEQ ID NO: 14 - DNA, 24 bp, Artificial sequence
  Primer
gatgaggtcaccattgtcaacatt

SEQ ID NO: 15 - DNA, 21 bp, Artificial sequence
  Primer
ggcgaaggcaatatcctgtct

SEQ ID NO: 16 - DNA, 22 bp, Artificial sequence
  Probe
tgaccaaccgcagcaatgcaca

SEQ ID NO: 17 - DNA, 19 bp, Artificial sequence
  Primer
gcgacttcaatttgccacg

SEQ ID NO: 18 - DNA, 21 bp, Artificial sequence
  Primer
ctgccttggatttgctcaatg

SEQ ID NO: 19 - DNA, 28 bp, Artificial sequence
  Probe
acccagccttcatccagttttatctgtt

SEQ ID NO: 20 - DNA, 22 bp, Artificial sequence
  Primer
ctctcaggagaccattgcatca

SEQ ID NO: 21 - DNA, 27 bp, Artificial sequence
  Primer
tcctgtctttgtactttcttcatttcc

SEQ ID NO: 22 - DNA, 24 bp, Artificial sequence
  Probe
ccgcacactggtggtccatgaaaa

SEQ ID NO: 23 - DNA, 19 bp, Artificial sequence
  Primer
gctggagtcgggctttacg

SEQ ID NO: 24 - DNA, 21 bp, Artificial sequence
  Primer
ataacgctttgcccgagactt

SEQ ID NO: 25 - DNA, 20 bp, Artificial sequence
  Probe
cgccggatggctctggacgt

SEQ ID NO: 26 - DNA, 23 bp, Artificial sequence
  Primer
ccagctacactcagaccaacaga

SEQ ID NO: 27 - DNA, 30 bp, Artificial sequence
  Primer
gaaaacggagaatctaataaaatcaatgac

SEQ ID NO: 28 - DNA, 26 bp, Artificial sequence
  Probe
cgtgacagccagcatcgaacatgaga

SEQ ID NO: 29 - DNA, 23 bp, Artificial sequence
  Primer
ccaataatgaagtgtggccagaa

SEQ ID NO: 30 - DNA, 20 bp, Artificial sequence
  Primer
gctaggcttcctggtttcca

SEQ ID NO: 31 - DNA, 31 bp, Artificial sequence
  Probe
tctttacaggactataagaatacatttccca

SEQ ID NO: 32 - DNA, 22 bp, Artificial sequence
  Primer
caagctctgtccgtatcgatca

SEQ ID NO: 33 - DNA, 25 bp, Artificial sequence
  Primer
tggacgataatcggaaaagtaatca

SEQ ID NO: 34 - DNA, 27 bp, Artificial sequence
  Probe
ctggatcgtgaacagttcaaccccgat

SEQ ID NO: 35 - DNA, 17 bp, Artificial sequence
  Primer
tggcgactgtcgaaccg

SEQ ID NO: 36 - DNA, 26 bp, Artificial sequence
  Primer
agattccgttttctcctcttctgtag

SEQ ID NO: 37 - DNA, 26 bp, Artificial sequence
  Probe
aaaccacccctactcctaatcccccg

SEQ ID NO: 38 - DNA, 18 bp, Artificial sequence
  Primer
tgcggccagcctgactag

SEQ ID NO: 39 - DNA, 23 bp, Artificial sequence
  Primer
cgtgattacacaccgactgagaa

SEQ ID NO: 40 - DNA, 20 bp, Artificial sequence
  Probe
ccccaccctgaggtcctgca

SEQ ID NO: 41 - DNA, 21 bp, Artificial sequence
  Primer
gctgcagaaccggagagattt

SEQ ID NO: 42 - DNA, 21 bp, Artificial sequence
  Primer
gggttaatgaggctcggtgtt

SEQ ID NO: 43 - DNA, 30 bp, Artificial sequence
  Probe
cagtgctctctggctaaagtcacggtcaaa

SEQ ID NO: 44 - DNA, 21 bp, Artificial sequence
  Primer
aaagcaaggtctccccacaag

SEQ ID NO: 45 - DNA, 24 bp, Artificial sequence
  Primer
tgaagggtctgtgctagatcaaaa

SEQ ID NO: 46 - DNA, 19 bp, Artificial sequence
  Probe
tgccacatcgccaccccgt

SEQ ID NO: 47 - DNA, 21 bp, Artificial sequence
  Primer
tggccatttccttctttcgtt

SEQ ID NO: 48 - DNA, 20 bp, Artificial sequence
  Primer
aaaacatcgctgataccagt

SEQ ID NO: 49 - DNA, 20 bp, Artificial sequence
  Synthetic oligonucleotide
ggcatatgcagataatgttc

SEQ ID NO: 50 - DNA, 20 bp, Artificial sequence
  Synthetic oligonucleotide
cgtcgtcgtcatcctcgtcc

SEQ ID NO: 51 - DNA, 20 bp, Artificial sequence
  Synthetic oligonucleotide
ctgctagcctctggatttga

SEQ ID NO: 52 - DNA, 20 bp, Artificial sequence
  Synthetic oligonucleotide
agcgagagctcgagactgag

SEQ ID NO: 53 - skipped

SEQ ID NO: 54 - DNA, 20 bp, Artificial sequence
  Synthetic oligonucleotide
taaagtgataatctttgtcg

SEQ ID NO: 55 - DNA, 20 bp, Artificial sequence
  Synthetic oligonucleotide
ctgccttcgtaattcattgc

SEQ ID NO: 56 - DNA, 20 bp, Artificial sequence
  Synthetic oligonucleotide
cggtcatgatgctgatccac

SEQ ID NO: 57 - DNA, 20 bp, Artificial sequence
  Synthetic oligonucleotide
ggttctggagcagatgatct

SEQ ID NO: 58 - DNA, 20 bp, Artificial sequence
  Synthetic oligonucleotide
ttttggcaaagtaatcgtcc

SEQ ID NO: 59 - DNA, 20 bp, Artificial sequence
  Synthetic oligonucleotide
cttctagagatttcatttca

SEQ ID NO: 60 - DNA, 20 bp, Artificial sequence
  Synthetic oligonucleotide
agacacatcggccacaccat

SEQ ID NO: 61 - DNA, 20 bp, Artificial sequence
  Synthetic oligonucleotide
acaccttcactggtccatta

SEQ ID NO: 62 - DNA, 20 bp, Artificial sequence
  Synthetic oligonucleotide
ctgctggaatgtttccactt

SEQ ID NO: 63 - DNA, 20 bp, Artificial sequence
  Synthetic oligonucleotide
tgtatgatctcctgccggtg

SEQ ID NO: 64 - DNA, 20 bp, Artificial sequence
  Synthetic oligonucleotide
agaacctgctggaactggcc

SEQ ID NO: 65 - DNA, 20 bp, Artificial sequence
  Synthetic oligonucleotide
aagagccgggtgtggtgcct

SEQ ID NO: 66 - DNA, 20 bp, Artificial sequence
  Synthetic oligonucleotide
tagaagtcgaactcggtggg

SEQ ID NO: 67 - DNA, 20 bp, Artificial sequence
  Synthetic oligonucleotide
tggtggtctcgcccgttact

SEQ ID NO: 68 - DNA, 20 bp, Artificial sequence
  Synthetic oligonucleotide
gccttggccagtgcttggtg

SEQ ID NO: 69 - DNA, 20 bp, Artificial sequence
  Synthetic oligonucleotide
gccaaggcgtgacatgatat

SEQ ID NO: 70 - DNA, 20 bp, Artificial sequence
  Synthetic oligonucleotide
ccatgatttcttaggcagct

SEQ ID NO: 71 - DNA, 20 bp, Artificial sequence
  Synthetic oligonucleotide
gccaacgactgattccataa

SEQ ID NO: 72 - DNA, 20 bp, Artificial sequence
  Synthetic oligonucleotide
gtactaggaaccccttcctc

SEQ ID NO: 73 - DNA, 20 bp, Artificial sequence
  Synthetic oligonucleotide
ggtctgaatctcctggcaaa

SEQ ID NO: 74 - DNA, 20 bp, Artificial sequence
  Synthetic oligonucleotide
tgtcatattcctggatcctt

SEQ ID NO: 75 - DNA, 20 bp, Artificial sequence
  Synthetic oligonucleotide
aagggtgcaaccgcttcgct

SEQ ID NO: 76 - DNA, 20 bp, Artificial sequence
  Synthetic oligonucleotide
gtcctcaggtgtcccatccc

SEQ ID NO: 77 - DNA, 20 bp, Artificial sequence
  Synthetic oligonucleotide
cggagcacgtacacccgaag

SEQ ID NO: 78 - DNA, 20 bp, Artificial sequence
  Synthetic oligonucleotide
tggcggccgcgggtgctgaa

SEQ ID NO: 79 - DNA, 20 bp, Artificial sequence
  Synthetic oligonucleotide
agatgagtccagaagaagcc

SEQ ID NO: 80 - DNA, 20 bp, Artificial sequence
  Synthetic oligonucleotide
tgaagcggacaagtggcgca

SEQ ID NO: 81 - DNA, 20 bp, Artificial sequence
  Synthetic oligonucleotide
ccatgatggcggcggagtgt

SEQ ID NO: 82 - DNA, 20 bp, Artificial sequence
  Synthetic oligonucleotide
tcctttggaggaggagccat

SEQ ID NO: 83 - DNA, 20 bp, Artificial sequence
  Synthetic oligonucleotide
cctcatcttcactatcttct

SEQ ID NO: 84 - DNA, 20 bp, Artificial sequence
  Synthetic oligonucleotide
tcttcttcatcttctgacat

SEQ ID NO: 85 - DNA, 20 bp, Artificial sequence
  Synthetic oligonucleotide
gacctcttctccactgctat

SEQ ID NO: 86 - DNA, 20 bp, Artificial sequence
  Synthetic oligonucleotide
tgcctttcttctgaggtatg

SEQ ID NO: 87 - DNA, 20 bp, Artificial sequence
  Synthetic oligonucleotide
gctgaggttgcagcagcctt

SEQ ID NO: 88 - DNA, 20 bp, Artificial sequence
  Synthetic oligonucleotide
ggtgtggcaactgcaacctt

SEQ ID NO: 89 - DNA, 20 bp, Artificial sequence
  Synthetic oligonucleotide
gagtgacagctgctttcttg

SEQ ID NO: 90 - DNA, 20 bp, Artificial sequence
  Synthetic oligonucleotide
ctgtcttcttggcaggtgtt

SEQ ID NO: 91 - DNA, 20 bp, Artificial sequence
  Synthetic oligonucleotide
gtaactgctttggctggtgt

SEQ ID NO: 92 - DNA, 20 bp, Artificial sequence
  Synthetic oligonucleotide
ggctcccttcttgccaggtg

SEQ ID NO: 93 - DNA, 20 bp, Artificial sequence
  Synthetic oligonucleotide
gctaccaatgctttgcctgg

SEQ ID NO: 94 - DNA, 20 bp, Artificial sequence
  Synthetic oligonucleotide
agcacccttcttaccaggag

SEQ ID NO: 95 - DNA, 20 bp, Artificial sequence
  Synthetic oligonucleotide
attcttgccattctttgccc

SEQ ID NO: 96 - DNA, 20 bp, Artificial sequence
  Synthetic oligonucleotide
atcactgtcttccttcttgg

SEQ ID NO: 97 - DNA, 20 bp, Artificial sequence
  Synthetic oligonucleotide
cactgtcatcatcctcctct

SEQ ID NO: 98 - DNA, 20 bp, Artificial sequence
  Synthetic oligonucleotide
ttcatcctcatcctcgtcct

SEQ ID NO: 99 - DNA, 20 bp, Artificial sequence
  Synthetic oligonucleotide
gctgctggttcaatttcatc

SEQ ID NO: 100 - DNA, 20 bp, Artificial sequence
  Synthetic oligonucleotide
gcagcagctgctgctttcat

SEQ ID NO: 101 - DNA, 20 bp, Artificial sequence
  Synthetic oligonucleotide
cttcgtcatcctcatcgtcc

SEQ ID NO: 102 - DNA, 20 bp, Artificial sequence
  Synthetic oligonucleotide
gtcatcgtcatcctcatcat

SEQ ID NO: 103 - DNA, 20 bp, Artificial sequence
  Synthetic oligonucleotide
cttcagagtcatcttcctca

SEQ ID NO: 104 - DNA, 20 bp, Artificial sequence
  Synthetic oligonucleotide
gtgtagtctccatagcttct

SEQ ID NO: 105 - DNA, 20 bp, Artificial sequence
  Synthetic oligonucleotide
gcagctttctttcctttggc

SEQ ID NO: 106 - DNA, 20 bp, Artificial sequence
  Synthetic oligonucleotide
ggctttcacaggaacaactt

SEQ ID NO: 107 - DNA, 20 bp, Artificial sequence
  Synthetic oligonucleotide
tcatcctcagccacgttctt

SEQ ID NO: 108 - DNA, 20 bp, Artificial sequence
  Synthetic oligonucleotide
catcatcttcatcatcttcg

SEQ ID NO: 109 - DNA, 20 bp, Artificial sequence
  Synthetic oligonucleotide
cctcctcatcatcttcatca

SEQ ID NO: 110 - DNA, 20 bp, Artificial sequence
  Synthetic oligonucleotide
tcttcctcctcctcttcttc

SEQ ID NO: 111 - DNA, 20 bp, Artificial sequence
  Synthetic oligonucleotide
ccaggtgcttctttgacagg

SEQ ID NO: 112 - DNA, 20 bp, Artificial sequence
  Synthetic oligonucleotide
gccatttccttctttcgttt

SEQ ID NO: 113 - DNA, 20 bp, Artificial sequence
  Synthetic oligonucleotide
ttcaggagctgctttctgtt

SEQ ID NO: 114 - DNA, 20 bp, Artificial sequence
  Synthetic oligonucleotide
ccttccactttctgtttctt

SEQ ID NO: 115 - DNA, 20 bp, Artificial sequence
  Synthetic oligonucleotide
gaaagccgtagtcggttctg

SEQ ID NO: 116 - DNA, 20 bp, Artificial sequence
  Synthetic oligonucleotide
ttaggtttccaacaaagaga

SEQ ID NO: 117 - DNA, 20 bp, Artificial sequence
  Synthetic oligonucleotide
tcaggagcagatttgttaaa

SEQ ID NO: 118 - DNA, 20 bp, Artificial sequence
  Synthetic oligonucleotide
ttctgacatccacaacagca

SEQ ID NO: 119 - DNA, 20 bp, Artificial sequence
  Synthetic oligonucleotide
cagattcaaaatccacataa

SEQ ID NO: 120 - DNA, 20 bp, Artificial sequence
  Synthetic oligonucleotide
acgctttctccaggtcttca

SEQ ID NO: 121 - DNA, 20 bp, Artificial sequence
  Synthetic oligonucleotide
actttcaaaccagtgagttc

SEQ ID NO: 122 - DNA, 20 bp, Artificial sequence
  Synthetic oligonucleotide
tagtttaatttcattgccaa

SEQ ID NO: 123 - DNA, 20 bp, Artificial sequence
  Synthetic oligonucleotide
tgtcttttccttttggtttc

SEQ ID NO: 124 - DNA, 20 bp, Artificial sequence
  Synthetic oligonucleotide
tcgcatctcgctctttctta

SEQ ID NO: 125 - DNA, 20 bp, Artificial sequence
  Synthetic oligonucleotide
tgactttgtaagggagattt

SEQ ID NO: 126 - DNA, 20 bp, Artificial sequence
  Synthetic oligonucleotide
acttctttcaattcatcctg

SEQ ID NO: 127 - DNA, 20 bp, Artificial sequence
  Synthetic oligonucleotide
gatctccgcagcatcttcaa

SEQ ID NO: 128 - DNA, 20 bp, Artificial sequence
  Synthetic oligonucleotide
cttttcccatccttgctgac

SEQ ID NO: 129 - DNA, 20 bp, Artificial sequence
  Synthetic oligonucleotide
tttctctgcatcagcttctg

SEQ ID NO: 130 - DNA, 20 bp, Artificial sequence
  Synthetic oligonucleotide
ttccctgcttttcttcaaag

SEQ ID NO: 131 - DNA, 20 bp, Artificial sequence
  Synthetic oligonucleotide
atagatcgcccatcgatctc

SEQ ID NO: 132 - DNA, 20 bp, Artificial sequence
  Synthetic oligonucleotide
ctctccagtatagtacaggg

SEQ ID NO: 133 - DNA, 20 bp, Artificial sequence
  Synthetic oligonucleotide
tagtcttgattttgaccttt

SEQ ID NO: 134 - DNA, 20 bp, Artificial sequence
  Synthetic oligonucleotide
agtgctattctttccacctc

SEQ ID NO: 135 - DNA, 20 bp, Artificial sequence
  Synthetic oligonucleotide
ggttgcttaaaaccagagtt

SEQ ID NO: 136 - DNA, 20 bp, Artificial sequence
  Synthetic oligonucleotide
ttctgttgcactgtaggaga

SEQ ID NO: 137 - DNA, 20 bp, Artificial sequence
  Synthetic oligonucleotide
aaatacttcctgaagagttt

SEQ ID NO: 138 - DNA, 20 bp, Artificial sequence
  Synthetic oligonucleotide
ttgataaaagttgctttctc

SEQ ID NO: 139 - DNA, 20 bp, Artificial sequence
  Synthetic oligonucleotide
cataccctttagatttgcca

SEQ ID NO: 140 - DNA, 20 bp, Artificial sequence
  Synthetic oligonucleotide
aatgaagcaaactctataaa

SEQ ID NO: 141 - DNA, 20 bp, Artificial sequence
  Synthetic oligonucleotide
agcttctttagcgtcttcga

SEQ ID NO: 142 - DNA, 20 bp, Artificial sequence
  Synthetic oligonucleotide
cccttttattacaggaattt

SEQ ID NO: 143 - DNA, 20 bp, Artificial sequence
  Synthetic oligonucleotide
tgattgctctgccctcaatt

SEQ ID NO: 144 - DNA, 20 bp, Artificial sequence
  Synthetic oligonucleotide
tgggtccttgcaactccagc

SEQ ID NO: 145 - DNA, 20 bp, Artificial sequence
  Synthetic oligonucleotide
tctggcattaggtgatcccc

SEQ ID NO: 146 - DNA, 20 bp, Artificial sequence
  Synthetic oligonucleotide
acagagttttggatggctgg

SEQ ID NO: 147 - DNA, 20 bp, Artificial sequence
  Synthetic oligonucleotide
tcctcagacaggcctttgac

SEQ ID NO: 148 - DNA, 20 bp, Artificial sequence
  Synthetic oligonucleotide
ctttaatgtctcttcagtgg

SEQ ID NO: 149 - DNA, 20 bp, Artificial sequence
  Synthetic oligonucleotide
gaacggagccgtcaaatgac

SEQ ID NO: 150 - DNA, 20 bp, Artificial sequence
  Synthetic oligonucleotide
cggtcagtaactatccttgc

SEQ ID NO: 151 - DNA, 20 bp, Artificial sequence
  Synthetic oligonucleotide
cagaagctattcaaacttcg

SEQ ID NO: 152 - DNA, 20 bp, Artificial sequence
  Synthetic oligonucleotide
ttgatcaggtaacagtaaaa

SEQ ID NO: 153 - DNA, 20 bp, Artificial sequence
  Synthetic oligonucleotide
atactgtcttggaatgtcct

SEQ ID NO: 154 - DNA, 20 bp, Artificial sequence
  Synthetic oligonucleotide
gatttccaaggagaccacag

SEQ ID NO: 155 - DNA, 20 bp, Artificial sequence
  Synthetic oligonucleotide
acacggtattgcccttgaaa

(19) United States
(12) Patent Application Publication
(10) Pub. No.: US 2021/0171945 A1
(43) Pub. Date: Jun. 10, 2021

(54) METHODS OF MODULATING ANTISENSE ACTIVITY

(71) Applicant: Ionis Pharmaceuticals, Inc., Carisbad, CA (US)

(72) Inventors: Xue-hai Liang, Del Mar, CA (US); Stanley T. Crooke, Carlsbad, CA (US)

(73) Assignee: Ionis Pharmaceuticals, Inc., Carlsbad, CA (US)

(21) Appl. No.: 16/762,734

(22) PCT Filed: Nov. 16, 2018
(86) PCT No.: PCT/US2018/061449

§ 371 Date: May 8, 2020

Related U.S. Application Data
(60) Provisional application No. 62/588,141, filed on Nov. 17, 2017.

(51) Int. Cl. C12N 15/113; A61K 45/06; A61K 31/7125

(52) U.S. Cl. CPC C12N 15/113; A61K 45/06; A61K 31/7125; C12N 2310/341; C12N 2310/3341; C12N 2310/11; C12N 2320/31; C12N 2320/50; C12N 2310/322; C12N 2310/315

(57) ABSTRACT

Disclosed herein are methods for increasing antisense activity by modulating translation. In certain embodiments, a compound comprising an antisense oligonucleotide is co-administered with an inhibitor of translation.

### SEQUENCE LISTING

The present application is being filed along with a Sequence Listing in electronic format. The Sequence Listing is provided as a file entitled CORE0146WOSEQ_ST25.txt, created Nov. 13, 2018, which is 36 Kb in size. The information in the electronic format of the sequence listing is incorporated herein by reference in its entirety.

### BACKGROUND

Most mRNAs are transcribed in the nucleus as pre-mRNAs, which are processed to mature mRNAs that are quickly exported to and enriched in the cytoplasm. During translation, a mRNA molecule can be translated simultaneously by more than one ribosome, forming poly-ribosomes (polysomes) that contain multiple 80S ribosomes per mRNA. Different mRNAs can be translated with variable efficiencies, which is mainly determined by the rate limiting step, translation initiation, and codon usage and mRNA structure affect the translation elongation rate. Efficiently translated mRNAs can be loaded with more 80S ribosomes per mRNA than the less efficiently translated mRNAs. Thus, the average distance between two adjacent ribosomes on a mRNA is mainly determined by the initiation efficiency.

RNase H1-dependent antisense oligonucleotides (ASOs) can trigger rapid degradation of mRNAs in the cytoplasm, where most mRNAs are translated under normal conditions. The effects of modulating translation on the activities of antisense oligonucleotides are unknown.

### SUMMARY OF THE INVENTION

Antisense oligonucleotides (ASOs) can act on translating mRNAs that are associated with ribosomes. Efficient translation of a target mRNA has a negative effect on activity of many ASOs that are complementary to the coding region of a target mRNA. Inhibition of translation increases the activity of such ASOs and does not increase the activity of ASOs targeting inefficiently or less efficiently translated mRNAs or non-coding RNAs. The efficiency of translation of a target mRNA can be determined using a variety of methods, such as those described in Schwanhausser et al. Nature. 473, 337-342 (2011) as well as methods described herein.

The present disclosure provides methods of identifying mRNA targets for ASO inhibition, methods of identifying target sites on target mRNAs, and methods of increasing ASO activity by modulating translation. In certain embodiments, the present disclosure provides methods comprising identifying target mRNAs that are slowly or inefficiently translated and inhibiting said target mRNAs with an ASO complementary to the coding region of the target mRNA. In certain embodiments, the present disclosure provides methods comprising administering an ASO and administering an inhibitor of translation. In certain embodiments, the present disclosure provides methods of inhibiting target mRNAs in rapidly proliferating cells by administrating an ASO complementary to the target mRNA and inhibiting translation in the cells.

### BRIEF DESCRIPTION OF FIGURES

FIG. 1 shows DNA sequencing from primer XL877 on the left and primer extension with primer XL877 on the right, in the presence and absence of CHX and DMS. The inset shows portions of the same gel with different exposure times.

FIG. 2 shows primer extension with primer XL845, at two different exposure times, in the presence and absence of CHX and DMS.

### DETAILED DESCRIPTION OF THE INVENTION

Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described.

### Definitions

As used herein, “2′-deoxynucleoside” means a nucleoside comprising 2′-H(H) ribosyl sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2′-fluoro” or “2′-F” means a 2′-F in place of the 2′-OH group of a ribosyl ring of a sugar moiety.

As used herein, “2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2-substituent group other than H or OH.

As used herein, “antisense activity” means any detectable and/or measurable change attributable to the hybridization of an antisense compound to its target nucleic acid. In certain embodiments, antisense activity is a decrease in the amount or expression of a target nucleic acid compared to target nucleic acid levels in the absence of the antisense compound.

As used herein, “antisense compound” means a compound comprising an antisense oligonucleotide and optionally one or more additional features, such as a conjugate group or terminal group.

As used herein, “antisense oligonucleotide” means an oligonucleotide having a nucleobase sequence that is at least partially complementary to a target nucleic acid.

As used herein, “ameliorate” in reference to a method means improvement in at least one symptom and/or measurable outcome relative to the same symptom or measurable outcome in the absence of or prior to performing the method. In certain embodiments, amelioration is the reduction in the severity or frequency of a symptom or the delayed onset or slowing of progression in the severity or frequency of a symptom and/or disease.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “cEt” or “constrained ethyl” means a ribosyl bicyclic sugar moiety wherein the second ring of the bicyclic sugar is formed via a bridge connecting the 4′-carbon and the 2′-carbon, wherein the bridge has the formula 4′-CH(CH₃)—O-2′, and wherein the methyl group of the bridge is in the S configuration.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “fully modified” in reference to a modified oligonucleotide means a modified oligonucleotide in which each sugar moiety is modified. “Uniformly modified” in reference to a modified oligonucleotide means a fully modified oligonucleotide in which each sugar moiety is the same. For example, the nucleosides of a uniformly modified oligonucleotide can each have a 2′-MOE modification but different nucleobase modifications, and the internucleoside linkages may be different.

As used herein, “gapmer” means an antisense oligonucleotide comprising an internal “gap” region having a plurality of nucleosides that support RNase H cleavage positioned between external “wing” regions having one or more nucleosides, wherein the nucleosides comprising the internal gap region are chemically distinct from the terminal wing nucleosides of the external wing regions.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting” or “inhibition” in refers to a partial or complete reduction. For example, inhibiting translation means a partial or complete reduction of translation, e.g., a decrease in the rate of translation or a decrease in the amount of protein produced via translation, and does not necessarily indicate a total elimination of translation.

As used herein, the terms “internucleoside linkage” means a group or bond that forms a covalent linkage between adjacent nucleosides in an oligonucleotide. As used herein “modified internucleoside linkage” means any internucleoside linkage other than a naturally occurring, phosphate internucleoside linkage. Non-phosphate linkages are referred to herein as modified internucleoside linkages. “Phosphorothioate linkage” means a modified phosphate linkage in which one of the non-bridging oxygen atoms is replaced with a sulfur atom. A phosphorothioate internucleoside linkage is a modified internucleoside linkage. Modified internucleoside linkages include linkages that comprise abasic nucleosides. As used herein, “abasic nucleoside” means a sugar moiety in an oligonucleotide that is not directly connected to a nucleobase. In certain embodiments, an abasic nucleoside is adjacent to one or two nucleosides in an oligonucleotide.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar” or “non-bicyclic modified sugar moiety” means a modified sugar moiety that comprises a modification, such as a substitutent, that does not form a bridge between two atoms of the sugar to form a second ring.

As used herein, “linked nucleosides” are nucleosides that are connected in a continuous sequence (i.e. no additional nucleosides are present between those that are linked).

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “modulation” means a perturbation of function, formation, activity, size, amount, or localization.

As used herein, “MOE” means methoxyethyl. “2′-MOE” means a 2′-OCH₂CH₂OCH₃ group in place of the 2′-OH group of a ribosyl ring of a sugar moiety.

As used herein, “motif” means the pattern of unmodified and/or modified sugar moieties, nucleobases, and/or internucleoside linkages, in an oligonucleotide.

As used herein, “naturally occurring” means found in nature.

As used herein, “nucleobase” means a naturally occurring nucleobase or a modified nucleobase. As used herein a “naturally occurring nucleobase” is adenine (A), thymine (T), cytosine (C), uracil (U), and guanine (G). As used herein, a modified nucleobase is a group of atoms capable of pairing with at least one naturally occurring nucleobase. A universal base is a nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety.

As used herein, “oligomeric compound” means a compound consisting of an oligonucleotide and optionally one or more additional features, such as a conjugate group or terminal group.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

As used herein “pharmaceutical composition” means a mixture of substances suitable for administering to a subject. For example, a pharmaceutical composition may comprise an antisense compound and a sterile aqueous solution. In certain embodiments, a pharmaceutical composition shows activity in free uptake assay in certain cell lines.

As used herein, “phosphorus moiety” means a group of atoms comprising a phosphorus atom. In certain embodiments, a phosphorus moiety comprises a mono-, di-, or tri-phosphate, or phosphorothioate.

As used herein “prodrug” means a therapeutic agent in a form outside the body that is converted to a differentform within the body or cells thereof. Typically conversion of a prodrug within the body is facilitated by the action of an enzymes (e.g., endogenous or viral enzyme) or chemicals present in cells or tissues and/or by physiologic conditions.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense oligonucleotides that act through RNase H.

As used herein, the term “single-stranded” in reference to an antisense compound and/or antisense oligonucleotide means such a compound consisting of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) moiety, as found in DNA (an “unmodified DNA sugar moiety”). As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars. As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid,” “target RNA,” “target RNA transcript” and “nucleic acid target” mean a nucleic acid that an antisense compound is designed to affect.

As used herein, “target region” means a portion of a target nucleic acid to which an antisense compound is designed to hybridize.

As used herein, “terminal group” means a chemical group or group of atoms that is covalently linked to a terminus of an oligonucleotide.

As used here, “terminal wing nucleoside” means a nucleoside that is located at the terminus of a wing segment of a gapmer. Any wing segment that comprises or consists of at least two nucleosides has two termini: one that immediately adjacent to the gap segment; and one that is at the end opposite the gap segment. Thus, any wing segment that comprises or consists of at least two nucleosides has two terminal nucleosides, one at each terminus.

### Certain Embodiments

The present disclosure includes but is not limited to the following embodiments.

I. Certain Oligonucleotides

In certain embodiments, the invention provides compounds, e.g., antisense compounds and oligomeric compounds, that comprise or consist of oligonucleotides that consist of linked nucleosides. Oligonucleotides, such as antisense oligonucleotides, may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

A. Certain Modified Nucleosides

Modified nucleosides comprise a modified sugar moiety or a modified nucleobase or both a modified sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including but not limited to substituents at the 2′, 4′, and/or 5′ positions. In certain embodiments, the furanosyl sugar moiety is a ribosyl sugar moiety. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.). In certain embodiments, a 2′-substituted nucleoside or 2′-non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl. In certain embodiments, a 2′-substituted nucleoside or 2′-non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a non-bridging 2′-substituent group selected from: F, OCH₃, and OCH₂CH₂OCH₃.

Nucleosides comprising modified sugar moieties, such as non-bicyclic modified sugar moieties, may be referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. In certain such embodiments, the furanose ring is a ribose ring. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Elayadi et al.; Wengel et al., U.S. Pat. No. 7,053,207; Imanishi et al., U.S. Pat. No. 6,268,490; Imanishi et al. U.S. Pat. No. 6,770,748; Imanishi et al., U.S. Pat. No. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the μ-D configuration.

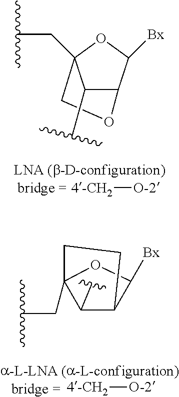

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the μ-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA.

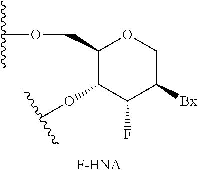

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3-fluoro tetrahydropyran), and nucleosides comprising additional modified THP compounds having the formula:

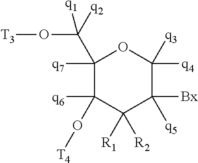

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

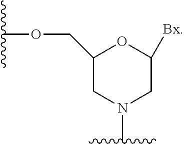

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides).

2. Certain Modified Nucleobases

In certain embodiments, oligonucleotides, e.g., antisense oligonucleotides, comprise one or more nucleoside comprising an unmodified nucleobase. In certain embodiments, modified oligonucleotides comprise one or more nucleoside comprising a modified nucleobase.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manohara et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

B. Certain Modified Internucleoside Linkages

In certain embodiments, nucleosides of oligonucleotides, including antisense oligonucleotides, may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS-P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

C. Certain Motifs

In certain embodiments, modified oligonucleotides, including modified antisense oligonucleotides, comprise one or more modified nucleoside comprising a modified sugar and/or a modified nucleobase. In certain embodiments, modified oligonucleotides, including modified antisense oligonucleotides, comprise one or more modified internucleoside linkage. In such embodiments, the modified, unmodified, and differently modified sugar moieties, nucleobases, and/or internucleoside linkages of a modified oligonucleotide, such as an antisense oligonucleotide, define a pattern or motif. In certain such embodiments, the patterns or motifs of sugar moieties, nucleobases, and internucleoside linkages are each independent of one another. Thus, a modified oligonucleotide, including an antisense oligonucleotide, may be described by its sugar motif, nucleobase motif and/or internucleoside linkage motif (as used herein, nucleobase motif describes the modifications to the nucleobases independent of the nucleobase sequence).

1. Certain Sugar Motifs

In certain embodiments, oligonucleotides, including antisense oligonucleotides, comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides, such as antisense oligonucleotides, comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least the sugar moieties of the terminal wing nucleosides of each of the wings differ from at least some of the sugar moieties of the nucleosides of the gap. In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5-wing differs from the sugar motif of the 3-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-5 nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiment, each nucleoside of the gap of a gapmer is an unmodified 2′-deoxynucleoside.

The nucleosides on the gap side of each wing/gap junction are unmodified 2′-deoxyribosyl nucleosides and the nucleosides on the wing sides of each wing/gap junction are modified nucleosides. In certain such embodiments, each nucleoside of the gap is an unmodified 2′-deoxyribosyl nucleoside. In certain such embodiments, each nucleoside of each wing is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain such embodiments, each nucleoside to the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

2. Certain Nucleobase Motifs

In certain embodiments, oligonucleotides, including antisense oligonucleotides, comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases are 5-methylcytosines.

In certain embodiments, modified oligonucleotides, such as modified antisense oligonucleotides, comprise a block of modified nucleobases. In certain such embodiments, the block is at the 3′-end of the oligonucleotide. In certain embodiments, the block is within 3 nucleosides of the 3′-end of the oligonucleotide. In certain embodiments, the block is at the 5′-end of the oligonucleotide. In certain embodiments, the block is within 3 nucleosides of the 5′-end of the oligonucleotide.

In certain embodiments, oligonucleotides, such as antisense oligonucleotides, having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

3. Certain Internucleoside Linkage Motifs

In certain embodiments, oligonucleotides, including antisense oligonucleotides, comprise modified and/or unmodified internucleoside linkages arranged along the oligonucleotide or region thereof in a defined pattern or motif. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified.

D. Certain Lengths

In certain embodiments, oligonucleotides, including antisense oligonucleotides, can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides

E. Certain Modified Oligonucleotides

In certain embodiments, the above modifications (sugar, nucleobase, internucleoside linkage) are incorporated into a modified oligonucleotide. In certain such embodiments, such modified oligonucleotides are antisense oligonucleotides. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Furthermore, in certain instances, an oligonucleotide is described by an overall length or range and by lengths or length ranges of two or more regions (e.g., regions of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists if of 15-20 linked nucleosides and has a sugar motif consisting of three regions, A, B, and C, wherein region A consists of 2-6 linked nucleosides having a specified sugar motif, region B consists of 6-10 linked nucleosides having a specified sugar motif, and region C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of the overall length of the modified oligonucleotide (20). Herein, if a description of an oligonucleotide is silent with respect to one or more parameter, such parameter is not limited. Thus, a modified oligonucleotide described only as having a gapmer sugar motif without further description may have any length, internucleoside linkage motif, and nucleobase motif Unless otherwise indicated, all modifications are independent of nucleobase sequence.

F. Nucleobase Sequence

In certain embodiments, oligonucleotides, such as antisense oligonucleotides, are further described by their nucleobase sequence. In certain embodiments, oligonucleotides have a nucleobase sequence that is complementary to a second oligonucleotide or a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

II. Certain Oligomeric Compounds

In certain embodiments, the invention provides oligomeric compounds, which consist of an oligonucleotide (e.g., a modified, unmodified, and/or antisense oligonucleotide) and optionally one or more conjugate groups and/or terminal groups. In certain embodiments, an oligomeric compound is also an antisense compound. In certain embodiments, an oligomeric compound is a component of an antisense compound. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain compounds comprising oligonucleotides, such as oligomeric compounds, the conjugate linker is a single chemical bond (i.e., the conjugate moiety is attached directly to an oligonucleotide through a single bond). In certain oligomeric compounds, a conjugate moiety is attached to an oligonucleotide via a more complex conjugate linker comprising one or more conjugate linker moeities, which are sub-units making up a conjugate linker.

In certain embodiments, the conjugate linker comprises a chain structure, such as a hydrocarbyl chain, or an oligomer of repeating units such as ethylene glycol, nucleosides, or amino acid units. In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

In certain embodiments, compounds of the invention are single-stranded. In certain embodiments, oligomeric compounds are paired with a second oligonucleotide or oligomeric compound to form a duplex, which is double-stranded.

III. Certain Antisense Compounds

In certain embodiments, the present invention provides antisense compounds, which comprise or consist of an oligomeric compound comprising an antisense oligonucleotide. In certain embodiments, antisense compounds are single-stranded. Such single-stranded antisense compounds typically comprise or consist of an oligomeric compound that comprises or consists of an antisense oligonucleotide and optionally a conjugate group. In certain embodiments, antisense compounds are double-stranded. Such double-stranded antisense compounds comprise a first oligomeric compound having a region complementary to a target nucleic acid and a second oligomeric compound having a region complementary to the first oligomeric compound. The first oligomeric compound of such double stranded antisense compounds typically comprises or consists of an antisense oligonucleotide and optionally a conjugate group. The oligonucleotide of the second oligomeric compound of such double-stranded antisense compound may be modified or unmodified. Either or both oligomeric compounds of a double-stranded antisense compound may comprise a conjugate group. The oligomeric compounds of double-stranded antisense compounds may include non-complementary overhanging nucleosides.

In certain embodiments, oligomeric compounds of antisense compounds are capable of hybridizing to a target nucleic acid, resulting in at least one antisense activity. In certain embodiments, antisense compounds selectively affect one or more target nucleic acid. Such selective antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, the invention provides antisense compounds that are sufficiently “DNA-like” to elicit RNase H activity. Further, in certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain such embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain such embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, and/or a phenotypic change in a cell or animal. In certain such embodiments, the target nucleic acid is a target mRNA.

IV. Certain Target Nucleic Acids

In certain embodiments, antisense compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is a mRNA. In certain such embodiments, the target region is entirely within an exon. In certain embodiments, the target region spans an exon/exon junction. In certain embodiments, antisense compounds are at least partially complementary to more than one target nucleic acid.

A. Complementarity/Mismatches to the Target Nucleic Acid

In certain embodiments, antisense compounds comprise antisense oligonucleotides that are complementary to the target nucleic acid over the entire length of the oligonucleotide. In certain embodiments, such oligonucleotides are 99% complementary to the target nucleic acid. In certain embodiments, such oligonucleotides are 95% complementary to the target nucleic acid. In certain embodiments, such oligonucleotides are 90% complementary to the target nucleic acid. In certain embodiments, such oligonucleotides are 85% complementary to the target nucleic acid. In certain embodiments, such oligonucleotides are 80% complementary to the target nucleic acid. In certain embodiments, antisense oligonucleotides are at least 80% complementary to the target nucleic acid over the entire length of the oligonucleotide and comprise a region that is 100% or fully complementary to a target nucleic acid. In certain such embodiments, the region of full complementarity is from 6 to 20 nucleobases in length. In certain such embodiments, the region of full complementarity is from 10 to 18 nucleobases in length. In certain such embodiments, the region of full complementarity is from 18 to 20 nucleobases in length.

In certain embodiments, oligonucleotides comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the antisense compound is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region.

V. Certain Pharmaceutical Compositions

In certain embodiments, the present invention provides pharmaceutical compositions comprising one or more antisense compound or a salt thereof. In certain such embodiments, the pharmaceutical composition comprises a suitable pharmaceutically acceptable diluent or carrier. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one antisense compound and sterile water. In certain embodiments, the sterile water is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile PBS. In certain embodiments, the sterile PBS is pharmaceutical grade PBS.

In certain embodiments, pharmaceutical compositions comprise one or more or antisense compound and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, antisense compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an antisense compound encompass any pharmaceutically acceptable salts of the antisense compound, esters of the antisense compound, or salts of such esters. In certain embodiments, pharmaceutical compositions comprising antisense compounds comprising one or more antisense oligonucleotide, upon administration to an animal, including a human, are capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an antisense compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

VI. Certain Combinations and Combination Therapies

In certain embodiments, methods provided herein comprise administering or contacting a cell with an antisense compound (first agent) and a compound that inhibits translation (second agent). In certain such embodiments, the second agent increases the activity of the first agent in a cell or individual relative to the activity of the first agent in a cell or individual in the absence of the second agent. In certain embodiments, co-administration of the first and second agents permits use of lower dosages than would be required to achieve a therapeutic or prophylactic effect if the agents were administered as independent therapies.

In certain embodiments, an antisense compound comprising or consisting of an antisense oligonucleotide is co-administered with one or more inhibitors of translation. In certain such embodiments, the antisense compound and one or more inhibitors of translation are administered at different times. In certain embodiments, the antisense compound and one or more inhibitors of translation are prepared together in a single formulation. In certain embodiments, the antisense compound and one or more inhibitors of translation are prepared separately. In certain embodiments, the one or more inhibitors of translation is a modified oligonucleotide complementary to the 5′-UTR of the target mRNA, puromycin, Rapamycin, Everolimus, Temsirolimus, Ridaforolimus, Hippuristanol, Homoharringtonine, cycloheximide, 4E1Rcat, lactimidomycin (LTM), or other inhibitor of translation, such as an inhibitor of translation intiation, translation elongation, or a direct inhibitor of the translation machinery (See, e.g., Bhat et al., Nat. Rev. Drug. Disc. 14, 261-278 (2015).)

In certain embodiments, an antisense compound comprising or consisting of an antisense oligonucleotide and one or more inhibitors of translation are used in combination treatment by administering the antisense compound and inhibitor of translation simultaneously, separately, or sequentially. In certain embodiments, they are formulated as a fixed dose combination product. In other embodiments, they are provided to the patient as separate units which can then either be taken simultaneously or serially (sequentially).

### Nonlimiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and other publications recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., antisense oligonucleotides) have one or more asymmetric center and thus give rise to enantiomers, diastereomers, and other stereoisomeric configurations that may be defined, in terms of absolute stereochemistry, as (R) or (S), as a or such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their racemic and optically pure forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### Example 1: Efficiency of mRNA Translation

Polysome profiles analyzed by sucrose gradient fractionation and RT-qPCR of the target mRNA of interest provide insight into the relative number of ribosomes actively translating a given mRNA molecule. This method has been described in Liang et al. Nat. Biotech., 34, 875-880 (2016).

Briefly, HeLa cells were grown to ˜80% confluency and treated with 100 μg/mL cycloheximide (CHX) for 15 minutes prior to lysis. Cell extracts were loaded onto a 7-47% sucrose gradient and 400 μL fractions were analyzed by RT-qPCR. NCL1 mRNA, PTEN mRNA, and 28S rRNA were detected with TaqMan primer probe sets, shown in Table 1 below. Elution of 28S rRNA peaks in the fractions containing 80S mono-ribosomes. Polysomes elute in later fractions, and the light polysomes that contain approximately 2-4 ribosomes per mRNA elute earlier than the heavy polysomes that contain approximately 5 or more ribosomes per mRNA. NCL mRNA is enriched in heavy polysomes, as most of the NCL mRNA eluted in the heavier polysome fractions, indicating that it is efficiently translated. PTEN mRNA is enriched in the 80S and lighter polysome fractions, indicating that it is inefficiently translated. Polysome analysis was completed with additional cellular mRNAs, as indicated in the tables below, and the mRNAs were classified as efficiently translated mRNA (NPM1, ANXA2, La, and SOD1) or inefficiently translated mRNA (Ago2, Drosha, ACP1, CDC2, CDK7, eIF4E, DPYSL).

TABLE 1
Primer Probe Sets

| | | | SEQ |
|---|---|---|---|
| Target | | Sequence (5′ to 3′) | ID NO |
| 28S | Forward | CAGGTCTCCAAGGTGAACAG | 2 |
| rRNA | Reverse | CTTAGAGCCAATCCTTATCCCG | 3 |
| | Probe | TCCCTTACCTACATTGTTCCAACATGCC | 4 |
| NCL1 | Forward | GCTTGGCTTCTTCTGGACTCA | 5 |
| | Reverse | TCGCGAGCTTCACCATGA | 6 |
| | Probe | CGCCACTTGTCCGCTTCACACTCC | 7 |
| PTEN | Forward | AATGGCTAAGTGAAGATGACAATCAT | 8 |
| | Reverse | TGCACATATCATTACACCAGTTCGT | 9 |
| | Probe | TTGCAGCAATTCACTGTAAAGCTGGAAAGG | 10 |
| NPM1 | Forward | TCCTGCGCGGTTGTTCTC | 11 |
| | Reverse | GGCGGCACGCACTTAGG | 12 |
| | Probe | CAGCGTTCTTTTATCTCCGTCCGCCT | 13 |
| ANXA2 | Forward | GATGAGGTCACCATTGTCAACATT | 14 |
| | Reverse | GGCGAAGGCAATATCCTGTCT | 15 |
| | Probe | TGACCAACCGCAGCAATGCACA | 16 |
| La | Forward | GCGACTTCAATTTGCCACG | 17 |
| | Reverse | CTGCCTTGGATTTGCTCAATG | 18 |
| | Probe | ACCCAGCCTTCATCCAGTTTTATCTGTT | 19 |
| SOD1 | Forward | CTCTCAGGAGACCATTGCATCA | 20 |
| | Reverse | TCCTGTCTTTGTACTTTCTTCATTTCC | 21 |
| | Probe | CCGCACACTGGTGGTCCATGAAAA | 22 |
| CDK7 | Forward | GCTGGAGTCGGGCTTTACG | 23 |
| | Reverse | ATAACGCTTTGCCCGAGACTT | 24 |
| | Probe | CGCCGGATGGCTCTGGACGT | 25 |
| Ago2 | Forward | CCAGCTACACTCAGACCAACAGA | 26 |
| | Reverse | GAAAACGGAGAATCTAATAAAATCAATGAC | 27 |
| | Probe | CGTGACAGCCAGCATCGAACATGAGA | 28 |
| CDC2 | Forward | CCAATAATGAAGTGTGGCCAGAA | 29 |
| | Reverse | GCTAGGCTTCCTGGTTTCCA | 30 |
| | Probe | TCTTTACAGGACTATAAGAATACATTTCCCA | 31 |
| Drosha | Forward | CAAGCTCTGTCCGTATCGATCA | 32 |
| | Reverse | TGGACGATAATCGGAAAAGTAATCA | 33 |
| | Probe | CTGGATCGTGAACAGTTCAACCCCGAT | 34 |
| eIF4E | Forward | TGGCGACTGTCGAACCG | 35 |
| | Reverse | AGATTCCGTTTTCTCCTCTTCTGTAG | 36 |
| | Probe | AAACCACCCCTACTCCTAATCCCCCG | 37 |
| ACP1 | Forward | TGCGGCCAGCCTGACTAG | 38 |
| | Reverse | CGTGATTACACACCGACTGAGAA | 39 |
| | Probe | CCCCACCCTGAGGTCCTGCA | 40 |
| DPYSL2 | Forward | GCTGCAGAACCGGAGAGATTT | 41 |
| | Reverse | GGGTTAATGAGGCTCGGTGTT | 42 |
| | Probe | CAGTGCTCTCTGGCTAAAGTCACGGTCAAA | 43 |

TABLE 2
Sucrose gradient fractions

| | NCL1 | PTEN | | |
|---|---|---|---|---|
| | mRNA | mRNA | 28S rRNA | |
| Fraction No. | (% total) | (% total) | (% total) | Elution region |
| F1 | 0.0 | 0.0 | 0.0 | |
| F2 | 0.0 | 0.0 | 0.0 | |
| F3 | 0.0 | 0.7 | 0.0 | |
| F4 | 0.1 | 1.2 | 0.0 | |
| F5 | 0.1 | 2.2 | 0.0 | |
| F6 | 0.4 | 3.8 | 0.1 | |
| F7 | 0.6 | 3.9 | 1.1 | |
| F8 | 0.4 | 2.1 | 2.7 | |
| F9 | 1.2 | 4.4 | 6.5 | 80S (mono-ribosomes) |
| F10 | 2.2 | 5.1 | 7.3 | |
| F11 | 2.7 | 8.9 | 15.0 | |
| F12 | 3.0 | 7.3 | 5.3 | |
| F13 | 1.9 | 5.1 | 4.2 | |
| F14 | 2.9 | 6.7 | 2.1 | Polysomes (gradient |
| F15 | 3.0 | 7.0 | 3.4 | from light to heavy) |
| F16 | 3.9 | 6.1 | 2.6 | |
| F17 | 4.0 | 5.6 | 2.5 | |
| F18 | 5.1 | 6.7 | 3.9 | |
| F19 | 5.1 | 4.5 | 4.7 | |
| F20 | 6.3 | 4.3 | 5.2 | |
| F21 | 6.9 | 3.4 | 4.2 | |
| F22 | 7.3 | 3.0 | 5.7 | |
| F23 | 6.4 | 1.8 | 3.6 | |
| F24 | 7.6 | 1.4 | 5.7 | |
| F25 | 6.6 | 1.0 | 3.4 | |
| F26 | 7.1 | 0.9 | 3.6 | |
| F27 | 5.2 | 0.5 | 2.7 | |
| F28 | 4.3 | 0.7 | 2.0 | |
| F29 | 5.6 | 1.7 | 2.5 | |

TABLE 3a
Sucrose gradient fractions

| Fraction | NPM1 | ANXA2 | La | SOD1 | CDK7 | Ago2 | 28S | Elution |
|---|---|---|---|---|---|---|---|---|
| No. | mRNA | mRNA | mRNA | mRNA | mRNA | mRNA | rRNA | region |
| F1 | 0.0 | 0.0 | 0.0 | 0.0 | 0.0 | 0.0 | 0.0 | |
| F2 | 0.1 | 0.1 | 0.0 | 0.1 | 0.0 | 0.1 | 0.0 | |
| F3 | 0.3 | 0.2 | 0.1 | 0.3 | 0.3 | 0.2 | 0.0 | |
| F4 | 0.9 | 0.2 | 0.4 | 0.3 | 0.6 | 0.4 | 0.0 | |
| F5 | 1.6 | 0.4 | 1.7 | 0.4 | 1.2 | 1.4 | 0.1 | |
| F6 | 1.4 | 0.4 | 1.9 | 0.4 | 1.7 | 2.3 | 0.4 | |
| F7 | 1.1 | 1.0 | 2.1 | 0.4 | 2.2 | 2.7 | 3.4 | |
| F8 | 1.4 | 0.6 | 2.0 | 0.7 | 7.2 | 2.8 | 6.8 | |
| F9 | 2.4 | 1.5 | 3.6 | 2.1 | 9.3 | 3.8 | 11.9 | 80S |
| F10 | 2.2 | 1.4 | 3.8 | 2.1 | 9.8 | 4.1 | 11.1 | (mono- |
| F11 | 1.5 | 1.3 | 2.0 | 2.4 | 9.9 | 3.3 | 8.5 | ribosomes) |
| F12 | 1.4 | 2.5 | 1.9 | 5.4 | 8.4 | 2.9 | 6.2 | |
| F13 | 1.9 | 2.3 | 1.7 | 4.6 | 6.0 | 4.4 | 4.7 | |
| F14 | 3.0 | 3.1 | 1.9 | 5.4 | 4.2 | 5.6 | 4.1 | Polysomes |
| F15 | 4.1 | 3.0 | 2.5 | 4.9 | 2.8 | 6.0 | 3.5 | (gradient |
| F16 | 5.7 | 2.5 | 2.6 | 8.3 | 2.5 | 6.2 | 2.5 | from light |
| F17 | 8.0 | 2.0 | 3.2 | 8.6 | 4.7 | 5.8 | 1.9 | to heavy) |
| F18 | 9.1 | 2.4 | 4.1 | 6.5 | 3.6 | 6.1 | 2.1 | |
| F19 | 12.8 | 3.2 | 5.3 | 8.1 | 3.9 | 5.9 | 2.3 | |
| F20 | 13.2 | 7.4 | 7.1 | 6.9 | 4.0 | 6.5 | 2.3 | |
| F21 | 10.6 | 9.0 | 10.7 | 8.7 | 3.2 | 6.6 | 3.0 | |
| F22 | 6.9 | 13.4 | 10.6 | 7.6 | 3.5 | 6.1 | 3.6 | |
| F23 | 4.0 | 13.9 | 11.9 | 6.2 | 2.8 | 6.2 | 3.6 | |
| F24 | 2.6 | 13.0 | 8.1 | 3.7 | 2.9 | 3.8 | 3.8 | |
| F25 | 1.7 | 4.7 | 5.2 | 1.2 | 2.0 | 3.1 | 4.0 | |
| F26 | 0.9 | 4.7 | 3.0 | 1.6 | 1.2 | 1.9 | 3.7 | |
| F27 | 0.5 | 1.7 | 1.3 | 0.7 | 0.9 | 0.9 | 2.1 | |
| F28 | 0.7 | 1.3 | 1.4 | 0.6 | 0.6 | 0.9 | 1.8 | |
| F29 | n.d. | 3.0 | n.d. | 1.5 | 0.7 | n.d. | 2.6 | |

TABLE 3b
Sucrose gradient fractions

| Fraction | CDC2 | Drosha | eIF4E | ACP1 | DPYSL2 | 28S | Elution |
|---|---|---|---|---|---|---|---|
| No. | mRNA | mRNA | mRNA | mRNA | mRNA | rRNA | region |
| F1 | 0.0 | 0.0 | 0.0 | 0.0 | 0.0 | 0.0 | |
| F2 | 0.0 | 0.2 | 0.0 | 0.0 | 0.0 | 0.0 | |
| F3 | 0.7 | 0.8 | 0.1 | 0.4 | 0.4 | 0.0 | |
| F4 | 1.9 | 1.0 | 0.2 | 1.5 | 1.1 | 0.0 | |
| F5 | 3.2 | 1.4 | 0.2 | 2.7 | 1.3 | 0.1 | |
| F6 | 3.4 | 1.8 | 0.4 | 2.5 | 2.3 | 0.4 | |
| F7 | 3.3 | 3.1 | 0.3 | 2.3 | 4.1 | 3.4 | |
| F8 | 5.8 | 4.1 | 0.6 | 6.3 | 4.3 | 6.8 | |
| F9 | 8.0 | 4.9 | 3.0 | 9.5 | 8.4 | 11.9 | 80S |
| F10 | 9.0 | 5.3 | 4.3 | 11.1 | 3.6 | 11.1 | (mono- |
| F11 | 8.1 | 4.5 | 3.1 | 9.2 | 3.6 | 8.5 | ribosomes) |
| F12 | 7.0 | 3.0 | 17.5 | 7.9 | 3.7 | 6.2 | |
| F13 | 4.2 | 3.1 | 25.4 | 4.2 | 1.9 | 4.7 | |
| F14 | 2.3 | 3.4 | 12.6 | 5.6 | 3.5 | 4.1 | Polysomes |
| F15 | 2.9 | 3.5 | 10.1 | 4.9 | 3.6 | 3.5 | (gradient |
| F16 | 2.5 | 3.7 | 8.8 | 5.8 | 3.8 | 2.5 | from light |
| F17 | 2.6 | 3.2 | 3.4 | 4.0 | 1.8 | 1.9 | to heavy) |
| F18 | 3.4 | 3.7 | 0.6 | 4.2 | 2.8 | 2.1 | |
| F19 | 6.8 | 4.0 | 2.0 | 3.9 | 3.0 | 2.3 | |
| F20 | 5.7 | 5.0 | 0.6 | 3.2 | 4.9 | 2.3 | |
| F21 | 3.6 | 6.5 | 0.6 | 2.5 | 2.4 | 3.0 | |
| F22 | 4.7 | 6.3 | 1.0 | 2.7 | 2.8 | 3.6 | |
| F23 | 3.5 | 8.2 | 1.4 | 1.6 | 5.3 | 3.6 | |
| F24 | 3.3 | 6.5 | 1.6 | 1.8 | 4.6 | 3.8 | |
| F25 | 1.9 | 5.5 | 0.7 | 0.8 | 5.1 | 4.0 | |
| F26 | 1.2 | 3.7 | 0.6 | 0.6 | 6.2 | 3.7 | |
| F27 | 0.5 | 1.9 | 0.3 | 0.4 | 7.4 | 2.1 | |
| F28 | 0.2 | 1.9 | 0.3 | 0.3 | 3.1 | 1.8 | |
| F29 | 0.3 | n.d. | 0.2 | 0.2 | 4.8 | 2.6 | |

### Example 2: Effects of Translation Inhibitors on NCL1 and PTEN ASO Activities

Antisense oligonucleotides complementary to three target mRNAs were synthesized and tested. The antisense oligonucleotides in the table below are gapmers 20 nucleobases in length, wherein each central gap segment contains ten 2′-deoxynucleosides and is flanked by wing segments on the 3′ and 5′ ends, each containing five 2′-methoxyethyl (MOE) nucleosides. All internucleoside linkages are phosphorothioate linkages.

TABLE 4
Antisense oligonucleotides

| | | Target mRNA | | SEQ |
|---|---|---|---|---|
| Compound | Target | translation | | ID |
| No. | mRNA | status | Sequence | NO |
| 395254 | Malat1 | untranslated | GGCATATGCA | 49 |
| | | | GATAATGTTC | |
| 110080 | NCL1 | efficiently | CGTCGTCGTC | 50 |
| | | translated | ATCCTCGTCC | |
| 116847 | PTEN | not efficiently | CTGCTAGCCT | 51 |
| | | translated | CTGGATTTGA | |

The activities of the antisense oligonucleotides when administered in combination with translation inhibitors were measured in multiple cell lines. HeLa cells were seeded at ˜50% confluence, and transfected the next day with Lipofectamine 2000 for 2.5 hours with antisense oligonucleotides at doses indicated in the tables. Cells were then treated with 100 μg/mL cycloheximide (CHX), 20 μM 4E1Rcat, 20 μg/mL puromycin (thermoFisher), 625 nM lactimidomycin (LTM, Millipore) or a control solution (ethanol control for CHX, DMSO control for 4E1Rcat and LTM, or water control for puromycin) for an additional 1.5 hours. Cells were then harvested and RT-qPCR was used to determine target mRNA levels as indicated in the tables below. Primer probe sets described in Example 1 were used for NCL1 and PTEN mRNA. For Malat 1, primer probe set had the following sequences: Forward sequence: 5′-AAAGCAAGGTCTCCCCACAAG-3′ (SEQ ID: 44); reverse sequence: 5′-TGAAGGGTCTGTGCTAGATCAAAA-3′, (SEQ ID: 45); Probe sequence: 5′-TGCCACATCGCCACCCCGT-3′, (SEQ ID 46). Treatment with translation inhibitors significantly altered antisense activity of compound no. 110080 targeting NCL1, but did not affect antisense activity of compound no. 116847 targeting PTEN.

A431 cells were incubated with antisense oligonucleotides for 16 hours via free uptake, then treated with ethanol or 100 μg/mL CHX for 1.5 hours. RNA levels were analyzed as described above.

Hek293 cells were transfected with Lipofectamine 2000 for 2.5 hours with antisense oligonucleotides at doses indicated in the tables, then treated with ethanol or 100 μg/mL CHX for 1.5 hours. RNA levels were analyzed as described above.

### Tables 5a-c: Effects of Translation Inhibition on Malat 1 Antisense Activity in HeLa Cells

TABLE 5a

| | [Compound no. 395254] (nM) | | | | | |
|---|---|---|---|---|---|---|
| | 0.0 | 0.1 | 0.4 | 1.1 | 3.3 | 10.0 |
| Cell treatment | Malat 1 mRNA (% control) | | | | | |
| Ethanol | 100 | 51 | 33 | 18 | 12 | 9 |
| CHX (100 μg/mL in EtOH) | 100 | 47 | 33 | 20 | 17 | 11 |
| DMSO | 100 | 78 | 53 | 31 | 20 | 4 |
| 4E1Rcat (20 μM in DMSO) | 100 | 82 | 54 | 34 | 24 | 7 |

TABLE 5b

| | [Compound no. 395254] (nM) | | | | |
|---|---|---|---|---|---|
| | 0 | 2 | 4 | 8 | 16 |
| Cell treatment | Malat 1 mRNA (% control) | | | | |
| Water | 100 | 74 | 39 | 26 | 15 |
| Puromycin | 100 | 83 | 40 | 22 | 13 |

TABLE 5c

[Compound no. 395254] (nM)

| | | | | | |
|---|---|---|---|---|---|
| 0 | 1.3 | 2.5 | 5 | 10 | 20 |

| | |
|---|---|
| Cell treatment | Malat 1 mRNA (% control) |

| | | | | | | |
|---|---|---|---|---|---|---|
| DMSO | 100 | 45 | 37 | 31 | 18 | 16 |
| LTM (625 nM) | 100 | 49 | 44 | 32 | 18 | 15 |

### Tables 6a-b: Effects of Translation Inhibition on NCL1 Antisense Activity in HeLa Cells

TABLE 6a

| | [Compound no. 110080] (nM) | | | | | |
|---|---|---|---|---|---|---|
| | 0.0 | 0.7 | 2.2 | 6.7 | 20.0 | 60.0 |
| Cell treatment | NCL1 mRNA (% control) | | | | | |
| Ethanol | 100 | 82 | 54 | 29 | 20 | 11 |
| CHX (100 μg/mL in EtOH) | 100 | 48 | 28 | 17 | 12 | 10 |
| DMSO | 100 | 93 | 86 | 61 | 27 | 10 |
| 4E1Rcat | 100 | 85 | 64 | 31 | 14 | 10 |
| LTM (625 nM) | 100 | 59 | 51 | 33 | 21 | 6 |

TABLE 6b

[Compound no. 110080] (nM)

| | | | | | |
|---|---|---|---|---|---|
| 0.0 | 7.5 | 15 | 30 | 60 | 120 |

| | |
|---|---|
| Cell treatment | NCL1 mRNA (% control) |

| | | | | | | |
|---|---|---|---|---|---|---|
| water | 100 | 61 | 43 | 22 | 11 | 8 |
| puromycin | 100 | 29 | 19 | 9 | 4 | 3 |

### Tables 7a-b: Effect of Translation Inhibition on PTEN Antisense Activity in HeLa Cells

TABLE 7a

| | [Compound no. 116847] (nM) | | | | | |
|---|---|---|---|---|---|---|
| | 0.0 | 0.7 | 2.2 | 6.7 | 20.0 | 60.0 |
| Cell treatment | PTEN mRNA (% control) | | | | | |
| Ethanol | 100 | 97 | 80 | 51 | 31 | 20 |
| CHX (100 μg/mL in EtOH) | 100 | 100 | 88 | 62 | 36 | 23 |
| DMSO | 100 | 88 | 75 | 46 | 24 | 19 |
| 4E1Rcat | 100 | 95 | 80 | 62 | 39 | 19 |

TABLE 7b

[Compound no. 116847] (nM)

| | | | | | |
|---|---|---|---|---|---|
| 0.0 | 7.5 | 15 | 30 | 60 | 120 |

| | |
|---|---|
| Cell treatment | PTEN mRNA (% control) |

| | | | | | | |
|---|---|---|---|---|---|---|
| DMSO | 100 | 91 | 84 | 65 | 47 | 39 |
| LTM (625 nM) | 100 | 101 | 87 | 73 | 47 | 34 |
| water | 100 | 92 | 76 | 47 | 27 | 24 |
| puromycin | 100 | 86 | 74 | 60 | 30 | 29 |

TABLE 8
Effect of translation inhibition on Malat-1
antisense activity in A431 Cells

| | [Compound no. 395254] (nM) | | | | | |
|---|---|---|---|---|---|---|
| | 0 | 12 | 37 | 111 | 333 | 1000 |
| Cell treatment | Malat-1 mRNA (% control) | | | | | |
| Ethanol | 100 | 89 | 64 | 33 | 19 | 16 |
| CHX (100 μg/mL in EtOH) | 100 | 88 | 65 | 37 | 26 | 21 |

TABLE 9
Effect of translation inhibition on
NCL1 antisense activity in A431 Cells

| | [Compound no. 110080] (nM) | | | | | |
|---|---|---|---|---|---|---|
| | 0 | 123 | 370 | 1111 | 3333 | 10000 |
| Cell treatment | NCL1 mRNA (% control) | | | | | |
| Ethanol | 100 | 86 | 73 | 64 | 51 | 39 |
| CHX (100 μg/mL in EtOH) | 100 | 67 | 48 | 31 | 21 | 18 |

TABLE 10
Effect of translation inhibition on PTEN antisense activity in A431 Cells

| | [Compound no. 116847] (nM) | | | | | |
|---|---|---|---|---|---|---|
| | 0 | 1250 | 2500 | 5000 | 10000 | 20000 |
| Cell treatment | PTEN mRNA (% control) | | | | | |
| Ethanol | 100 | 69 | 62 | 65 | 56 | 52 |
| CHX (100 μg/mL in EtOH) | 100 | 72 | 59 | 61 | 57 | 51 |

TABLE 11
Effect of translation inhibition on Malat-1 antisense activity in
Hek293 Cells

| | [Compound no. 395254] (nM) | | | | |
|---|---|---|---|---|---|
| | 0 | 0.25 | 0.5 | 1 | 2 |
| Cell treatment | Malat-1 mRNA (% control) | | | | |
| Ethanol | 100 | 79 | 63 | 53 | 26 |
| CHX (100 μg/mL in EtOH) | 100 | 78 | 58 | 52 | 21 |

TABLE 12
Effect of translation inhibition on NCL1
antisense activity in Hek293 Cells

| | [Compound no. 110080] (nM) | | | | | |
|---|---|---|---|---|---|---|
| | 0 | 0.7 | 2.2 | 6.7 | 20 | 60 |
| Cell treatment | NCL1 mRNA (% control) | | | | | |
| Ethanol | 100 | 83 | 59 | 45 | 30 | 21 |
| CHX (100 μg/mL in EtOH) | 100 | 60 | 41 | 36 | 23 | 16 |

TABLE 13
Effect of translation inhibition on PTEN
antisense activity in Hek293 Cells

| | [Compound no. 116847] (nM) | | | | | |
|---|---|---|---|---|---|---|
| | 0 | 0.7 | 2.2 | 6.7 | 20 | 60 |
| Cell treatment | PTEN mRNA (% control) | | | | | |
| Ethanol | 100 | 102 | 76 | 50 | 34 | 16 |
| CHX (100 μg/mL in EtOH) | 100 | 97 | 83 | 52 | 29 | 21 |

### Example 3: Effects of an ASO Translation Inhibitor on NCL1 ASO Activity

A uniformly modified 2′-MOE oligonucleotide was synthesized for use in specifically blocking translation NCL1 by hybridizing to the 5′ UTR of NCL1 mRNA. Compound no. 877860 is 100% complementary to the 5′ UTR of NCL1 and has the sequence AGCGAGAGCTCGAGACTGAG (SEQ ID NO: 52). HeLa cells were transfected with compound no. 877860 or a control oligonucleotide complementary to NPM1 with Lipofectamine 2000 at 40 nM for 16 hours. A gapmer ASO listed in the table below was then transfected for 4 hours. Cells were lysed and RNA analyzed as in the Examples above. Cell lysate was also used to run a Western blot for NCL1 protein levels, which were detected with ab13541 (Abam) followed by anti-mouse-HRP (170-6516, Bio-Rad). Protein levels were normalized to TCP1β, detected by ab92746 (Abcam) followed by anti-rabbit-HRP (170-6515, Bio-Rad). Compound no. 877860 targeted to the 5′ UTR of NCL1 reduced levels of NCL1 protein and increased the activity of compound no. 110080, while similar effects were not observed for ASOs targeted to PTEN or NPM1, or for treatment with 877862.

TABLE 14
Effect of translation inhibition of NCL mRNA
on antisense activities in HeLa Cells

| Gapmer | | | | | | | | |
|---|---|---|---|---|---|---|---|---|
| ASO | | | [Gapmer] (nM) | | | | | |
| Compound | Target | Uniformly | 0 | 0.7 | 2.2 | 6.7 | 20 | 60 |
| No. | mRNA | modified ASO | mRNA level (% control) | | | | | |
| 110080 | NCL1 | 877860 | 100 | 35 | 22 | 17 | 12 | 10 |
| | | Control | 100 | 62 | 44 | 24 | 12 | 9 |
| 116847 | PTEN | 877860 | 100 | 87 | 73 | 53 | 37 | 37 |
| | | Control | 100 | 84 | 78 | 60 | 39 | 38 |

### Example 4: Effects of Translation Inhibitors on ASOs Targeting Efficiently Translated mRNAs

Antisense oligonucleotides shown in the table below are gapmers 20 nucleobases in length, wherein each central gap segment contains ten 2′-deoxynucleosides and is flanked by wing segments on the 3′ and 5′ ends, each containing five 2′-MOE nucleosides. Compound 611458 contains phosphorothioate and phosphate internucleoside linkages of the following motif from 5′ to 3′: sooos sssss sssss ooos, wherein “s” represents a phosphothioate linkage and “o” represents a phosphate linkage. All of the internucleoside linkages of the remaining compounds are phosphothioate linkages. All of the cytosines in each of the antisense oligonucleotides are 5-methylcytosines.

TABLE 15
Antisense oligonucleotides

| Compound | | | SEQ |
|---|---|---|---|
| No. | Target | Sequence | ID NO |
| 573658 | NPM1 | TAAAGTGATAATCTTTGTCG | 54 |
| 573657 | NPM1 | CTGCCTTCGTAATTCATTGC | 55 |
| 344980 | ANXA2 | CGGTCATGATGCTGATCCAC | 56 |
| 344968 | ANXA2 | GGTTCTGGAGCAGATGATCT | 57 |
| 286529 | La | TTTTGGCAAAGTAATCGTCC | 58 |
| 286532 | La | CTTCTAGAGATTTCATTTCA | 59 |
| 489505 | SOD1 | AGACACATCGGCCACACCAT | 60 |
| 611458 | SOD1 | ACACCTTCACTGGTCCATTA | 61 |

HeLa cells were transfected with an antisense oligonucleotide followed by treatment with CHX as described in Example 2. RT-PCR was used to determine antisense activity of each oligonucleotide in ethanol treated cells compared to translation-inhibited CHX treated cells, using the primer probe sets described above. The results show that the activities of these antisense oligonucleotides was increased when translation was inhibited.

TABLE 16
Effect of translation inhibition on
NPM1 antisense activity in HeLa Cells

| ASO | | [ASO] (nM) | | | | | |
|---|---|---|---|---|---|---|---|
| compound | | 0 | 0.74 | 2.22 | 6.67 | 20 | 60 |
| No. | Cell treatment | NPM1 mRNA (% control) | | | | | |
| 573658 | Ethanol | 100 | 100 | 92 | 72 | 51 | 12 |
| | CHX (100 μg/mL | 100 | 86 | 60 | 42 | 17 | 6 |
| | in EtOH) | | | | | | |
| 573657 | Ethanol | 100 | 105 | 105 | 106 | 86 | 57 |
| | CHX (100 μg/mL | 100 | 90 | 94 | 87 | 63 | 33 |
| | in EtOH) | | | | | | |

TABLE 17a
Effect of translation inhibition on ANXA2
antisense activity in HeLa Cells

| ASO | | [ASO] (nM) | | | | | |
|---|---|---|---|---|---|---|---|
| compound | | 0 | 0.74 | 2.22 | 6.67 | 20 | 60 |
| No. | Cell treatment | ANXA2 mRNA (% control) | | | | | |
| 344980 | Ethanol | 100 | 101 | 93 | 80 | 55 | 18 |
| | CHX (100 μg/mL | 100 | 95 | 85 | 58 | 27 | 8 |
| | in EtOH) | | | | | | |

TABLE 17b
Effect of translation inhibition on ANXA2
antisense activity in HeLa Cells

| ASO | | [ASO] (nM) | | | | | |
|---|---|---|---|---|---|---|---|
| compound | | 0 | 5 | 10 | 20 | 40 | 80 |
| No. | Cell treatment | ANXA2 mRNA (% control) | | | | | |
| 344968 | Ethanol | 100 | 115 | 105 | 94 | 71 | 56 |
| | CHX (100 μg/mL | 100 | 109 | 98 | 72 | 45 | 39 |
| | in EtOH) | | | | | | |

TABLE 18a
Effect of translation inhibition on
La antisense activity in HeLa Cells

| ASO | | [ASO] (nM) | | | | | |
|---|---|---|---|---|---|---|---|
| compound | | 0 | 0.74 | 2.22 | 6.67 | 20 | 60 |
| No. | Cell treatment | La mRNA (% control) | | | | | |
| 286529 | Ethanol | 100 | 87 | 77 | 46 | 22 | 5 |
| | CHX (100 μg/mL | 100 | 77 | 58 | 31 | 10 | 1 |
| | in EtOH) | | | | | | |

TABLE 18b
Effect of translation inhibition on
La antisense activity in HeLa Cells

| ASO | | [ASO] (nM) | | | | | |
|---|---|---|---|---|---|---|---|
| compound | | 0 | 7.5 | 15 | 30 | 60 | 120 |
| No. | Cell treatment | La mRNA (% control) | | | | | |
| 286532 | Ethanol | 100 | 92 | 93 | 68 | 56 | 41 |
| | CHX (100 μg/mL | 100 | 97 | 84 | 47 | 32 | 20 |
| | in EtOH) | | | | | | |

TABLE 19a
Effect of translation inhibition on
SOD1 antisense activity in HeLa Cells

| ASO | | [ASO] (nM) | | | | | |
|---|---|---|---|---|---|---|---|
| compound | | 0 | 0.74 | 2.22 | 6.67 | 20 | 60 |
| No. | Cell treatment | SOD1 mRNA (% control) | | | | | |
| 489505 | Ethanol | 100 | 95 | 98 | 94 | 65 | 44 |
| | CHX (100 μg/mL | 100 | 90 | 89 | 74 | 47 | 21 |
| | in EtOH) | | | | | | |

TABLE 19b
Effect of translation inhibition on
SOD1 antisense activity in HeLa Cells

| ASO | | [ASO] (nM) | | | | | |
|---|---|---|---|---|---|---|---|
| compound | | 0 | 7.5 | 15 | 30 | 60 | 120 |
| No. | Cell treatment | SOD1 mRNA (% control) | | | | | |
| 611458 | Ethanol | 100 | 95 | 93 | 66 | 33 | 24 |
| | CHX (100 μg/mL | 100 | 92 | 78 | 35 | 17 | 11 |
| | in EtOH) | | | | | | |

### Example 5: Effects of Translation Inhibitors on ASOs Targeting Inefficiently Translated mRNAs

Antisense oligonucleotides shown in the table below are gapmers 20 nucleobases in length, wherein each central gap segment contains ten 2′-deoxynucleosides and is flanked by wing segments on the 3′ and 5′ ends each containing five 2′-MOE nucleosides. All of the internucleoside linkages in the antisense oligonucleotides are phosphorothioate linkages, and all of the cytosines are 5-methylcytosines.

TABLE 20
Antisense oligonucleotides

| Compound | mRNA | | SEQ ID |
|---|---|---|---|
| No. | target | Sequence | NO |
| 136764 | Ago2 | CTGCTGGAATGTTTCCACTT | 62 |
| 136754 | Ago2 | TGTATGATCTCCTGCCGGTG | 63 |
| 136758 | Ago2 | AGAACCTGCTGGAACTGGCC | 64 |
| 136762 | Ago2 | AAGAGCCGGGTGTGGTGCCT | 65 |
| 136766 | Ago2 | TAGAAGTCGAACTCGGTGGG | 66 |
| 136775 | Ago2 | TGGTGGTCTCGCCCGTTACT | 67 |
| 136777 | Ago2 | GCCTTGGCCAGTGCTTGGTG | 68 |
| 25691 | Drosha | GCCAAGGCGTGACATGATAT | 69 |
| 356752 | ACP1 | CCATGATTTCTTAGGCAGCT | 70 |
| 356789 | ACP1 | GCCAACGACTGATTCCATAA | 71 |
| 207215 | CDC2 | GTACTAGGAACCCCTTCCTC | 72 |
| 169350 | CDK7 | GGTCTGAATCTCCTGGCAAA | 73 |
| 1803750 | eIF4E | TGTCATATTCCTGGATCCTT | 74 |
| 138020 | DPYSL2 | AAGGGTGCAACCGCTTCGCT | 75 |
| 138056 | DPYSL2 | GTCCTCAGGTGTCCCATCCC | 76 |

HeLa cells were transfected with an antisense oligonucleotide followed by treatment with CHX as described in Example 2. RT-qPCR was used to determine antisense activity of each oligonucleotide in ethanol treated cells compared to translation-inhibited CHX treated cells, using the primer probe sets described above. The results show that the activities of these antisense oligonucleotides targeting inefficiently translated mRNAs were not affected when translation was inhibited.

TABLE 21
Effect of translation inhibition on
Ago2 antisense activity in HeLa Cells

| ASO | | [ASO] (nM) | | | | | |
|---|---|---|---|---|---|---|---|
| compound | | 0 | 0.7 | 2.2 | 6.7 | 20 | 60 |
| No. | Cell treatment | Ago2 mRNA level (% control) | | | | | |
| 136764 | Ethanol | 100 | 83 | 66 | 44 | 26 | 13 |
| | CHX (100 μg/mL in | 100 | 84 | 59 | 40 | 25 | 12 |
| | EtOH) | | | | | | |
| 136754 | Ethanol | 100 | 95 | 109 | 87 | 44 | 23 |
| | CHX (100 μg/mL in | 100 | 95 | 91 | 92 | 48 | 21 |
| | EtOH) | | | | | | |
| 136758 | Ethanol | 100 | 96 | 97 | 98 | 52 | 27 |
| | CHX (100 μg/mL in | 100 | 107 | 103 | 92 | 51 | 21 |
| | EtOH) | | | | | | |
| 136762 | Ethanol | 100 | 119 | 106 | 108 | 85 | 51 |
| | CHX (100 μg/mL in | 100 | 110 | 97 | 101 | 79 | 53 |
| | EtOH) | | | | | | |
| 136766 | Ethanol | 100 | 94 | 89 | 91 | 48 | 28 |
| | CHX (100 μg/mL in | 100 | 98 | 97 | 92 | 43 | 27 |
| | EtOH) | | | | | | |
| 136775 | Ethanol | 100 | 94 | 98 | 91 | 45 | 28 |
| | CHX (100 μg/mL in | 100 | 105 | 94 | 97 | 46 | 21 |
| | EtOH) | | | | | | |
| 136777 | Ethanol | 100 | 109 | 102 | 106 | 54 | 31 |
| | CHX (100 μg/mL in | 100 | 95 | 103 | 101 | 46 | 29 |
| | EtOH) | | | | | | |

TABLE 22
Effect of translation inhibition on antisense activity in HeLa cells

| ASO | | | [ASO] (nM) | | | | | |
|---|---|---|---|---|---|---|---|---|
| compound | mRNA | | 0 | 0.7 | 2.2 | 6.7 | 20 | 60 |
| No. | target | Cell treatment | mRNA level (% control) | | | | | |
| 25691 | Drosha | Ethanol | 100 | 96 | 86 | 74 | 57 | 39 |
| | | CHX (100 μg/mL in | 100 | 95 | 80 | 73 | 56 | 38 |
| | | EtOH) | | | | | | |
| 356752 | ACP1 | Ethanol | 100 | 72 | 50 | 40 | 30 | 25 |
| | | CHX (100 μg/mL in | 100 | 78 | 57 | 50 | 37 | 23 |
| | | EtOH) | | | | | | |
| 356789 | ACP1 | Ethanol | 100 | 72 | 50 | 40 | 30 | 25 |
| | | CHX (100 μg/mL in | 100 | 89 | 86 | 51 | 37 | 23 |
| | | EtOH) | | | | | | |
| 207215 | CDC2 | Ethanol | 100 | 92 | 92 | 77 | 61 | 28 |
| | | CHX (100 μg/mL in | 100 | 95 | 89 | 86 | 60 | 31 |
| | | EtOH) | | | | | | |
| 169350 | CDK7 | Ethanol | 100 | 80 | 64 | 39 | 27 | 16 |
| | | CHX (100 μg/mL in | 100 | 82 | 57 | 50 | 38 | 24 |
| | | EtOH) | | | | | | |
| 1803750 | eIF4E | Ethanol | 100 | 78 | 62 | 50 | 30 | 11 |
| | | CHX (100 μg/mL in | 100 | 96 | 84 | 61 | 50 | 28 |
| | | EtOH) | | | | | | |
| 138020 | DPYSL2 | Ethanol | 100 | 75 | 51 | 28 | 17 | 9 |
| | | CHX (100 μg/mL in | 100 | 88 | 80 | 58 | 36 | 12 |
| | | EtOH) | | | | | | |
| 138056 | DPYSL2 | Ethanol | 100 | 86 | 73 | 62 | 44 | 43 |
| | | CHX (100 μg/mL in | 100 | 103 | 87 | 87 | 79 | 54 |
| | | EtOH) | | | | | | |

### Example 6: Effect of Translation Inhibition on Activities of Antisense Oligonucleotides Targeting NCL1

The effects of translation inhibition on the antisense activities of antisense oligonucleotides complementary to various sites along NCL1 mRNA were tested in HeLa cells. The antisense oligonucleotides in the table below are gapmers 20 nucleobases in length, wherein each central gap segment contains ten 2′-deoxynucleosides and is flanked by wing segments on the 3′ and 5′ ends each containing five 2′-MOE nucleosides. “Start Site” indicates the 5′-most nucleoside to which the gapmer is complementary in the target mRNA sequence. “Stop Site” indicates the 3′-most nucleoside to which the gapmer is complementary in the target mRNA sequence. The antisense oligonucleotides are 1000 complementary to GenBank accession number NM_005381.2, SEQ ID NO: 1.

TABLE 23
Antisense oligonucleotides

| Compound | | start | stop | SEQ ID |
|---|---|---|---|---|
| No. | Sequence | site | site | NO |
| 110049 | CGGAGCACGTACACCCGAAG | 31 | 50 | 77 |
| 110050 | TGGCGGCCGCGGGTGCTGAA | 56 | 75 | 78 |
| 110051 | AGATGAGTCCAGAAGAAGCC | 88 | 107 | 79 |
| 110052 | TGAAGCGGACAAGTGGCGCA | 107 | 126 | 80 |
| 110053 | CCATGATGGCGGCGGAGTGT | 126 | 145 | 81 |
| 110054 | TCCTTTGGAGGAGGAGCCAT | 190 | 209 | 82 |
| 110055 | CCTCATCTTCACTATCTTCT | 216 | 235 | 83 |
| 110056 | TCTTCTTCATCTTCTGACAT | 238 | 257 | 84 |
| 110057 | GACCTCTTCTCCACTGCTAT | 260 | 279 | 85 |
| 110058 | TGCCTTTCTTCTGAGGTATG | 282 | 301 | 86 |
| 110059 | GCTGAGGTTGCAGCAGCCTT | 304 | 323 | 87 |
| 110060 | GGTGTGGCAACTGCAACCTT | 352 | 371 | 88 |
| 110061 | GAGTGACAGCTGCTTTCTTG | 375 | 394 | 89 |
| 110062 | CTGTCTTCTTGGCAGGTGTT | 414 | 433 | 90 |
| 110063 | GTAACTGCTTTGGCTGGTGT | 436 | 455 | 91 |
| 110064 | GGCTCCCTTCTTGCCAGGTG | 458 | 477 | 92 |
| 110065 | GCTACCAATGCTTTGCCTGG | 481 | 500 | 93 |
| 110066 | AGCACCCTTCTTACCAGGAG | 503 | 522 | 94 |
| 110067 | ATTCTTGCCATTCTTTGCCC | 539 | 558 | 95 |
| 110068 | ATCACTGTCTTCCTTCTTGG | 560 | 579 | 96 |
| 110069 | CACTGTCATCATCCTCCTCT | 582 | 601 | 97 |
| 110070 | TTCATCCTCATCCTCGTCCT | 623 | 642 | 98 |
| 110071 | GCTGCTGGTTCAATTTCATC | 643 | 662 | 99 |
| 110072 | GCAGCAGCTGCTGCTTTCAT | 664 | 683 | 100 |
| 110073 | CTTCGTCATCCTCATCGTCC | 702 | 721 | 101 |
| 110074 | GTCATCGTCATCCTCATCAT | 722 | 741 | 102 |
| 110075 | CTTCAGAGTCATCTTCCTCA | 744 | 763 | 103 |
| 110076 | GTGTAGTCTCCATAGCTTCT | 765 | 784 | 104 |
| 110077 | GCAGCTTTCTTTCCTTTGGC | 787 | 806 | 105 |
| 110078 | GGCTTTCACAGGAACAACTT | 809 | 828 | 106 |
| 110079 | TCATCCTCAGCCACGTTCTT | 829 | 848 | 107 |
| 110080 | CGTCGTCGTCATCCTCGTCC | 870 | 889 | 50 |
| 110081 | CATCATCTTCATCATCTTCG | 891 | 910 | 108 |
| 110082 | CCTCCTCATCATCTTCATCA | 912 | 931 | 109 |
| 110083 | TCTTCCTCCTCCTCTTCTTC | 934 | 953 | 110 |
| 110084 | CCAGGTGCTTCTTTGACAGG | 955 | 974 | 111 |
| 110085 | GCCATTTCCTTCTTTCGTTT | 976 | 995 | 112 |
| 110086 | TTCAGGAGCTGCTTTCTGTT | 998 | 1017 | 113 |
| 110087 | CCTTCCACTTTCTGTTTCTT | 1021 | 1040 | 114 |
| 110088 | GAAAGCCGTAGTCGGTTCTG | 1043 | 1062 | 115 |
| 110089 | TTAGGTTTCCAACAAAGAGA | 1065 | 1084 | 116 |
| 110090 | TCAGGAGCAGATTTGTTAAA | 1087 | 1106 | 117 |
| 110091 | TTCTGACATCCACAACAGCA | 1149 | 1168 | 118 |
| 110092 | CAGATTCAAAATCCACATAA | 1191 | 1210 | 119 |
| 110093 | ACGCTTTCTCCAGGTCTTCA | 1212 | 1231 | 120 |
| 110094 | ACTTTCAAACCAGTGAGTTC | 1234 | 1253 | 121 |
| 110095 | TAGTTTAATTTCATTGCCAA | 1256 | 1275 | 122 |
| 110096 | TGTCTTTTCCTTTTGGTTTC | 1278 | 1297 | 123 |
| 110097 | TCGCATCTCGCTCTTTCTTA | 1299 | 1318 | 124 |
| 110098 | TGACTTTGTAAGGGAGATTT | 1335 | 1354 | 125 |
| 110099 | ACTTCTTTCAATTCATCCTG | 1357 | 1376 | 126 |
| 110100 | GATCTCCGCAGCATCTTCAA | 1379 | 1398 | 127 |
| 110101 | CTTTTCCCATCCTTGCTGAC | 1405 | 1424 | 128 |
| 110102 | TTTCTCTGCATCAGCTTCTG | 1454 | 1473 | 129 |
| 110103 | TTCCCTGCTTTTCTTCAAAG | 1476 | 1495 | 130 |
| 110104 | ATAGATCGCCCATCGATCTC | 1498 | 1517 | 131 |
| 110105 | CTCTCCAGTATAGTACAGGG | 1520 | 1539 | 132 |
| 110106 | TAGTCTTGATTTTGACCTTT | 1540 | 1559 | 133 |
| 110107 | AGTGCTATTCTTTCCACCTC | 1562 | 1581 | 134 |
| 110108 | GGTTGCTTAAAACCAGAGTT | 1599 | 1618 | 135 |
| 110109 | TTCTGTTGCACTGTAGGAGA | 1619 | 1638 | 136 |
| 110110 | AAATACTTCCTGAAGAGTTT | 1640 | 1659 | 137 |
| 110111 | TTGATAAAAGTTGCTTTCTC | 1660 | 1679 | 138 |
| 110112 | CATACCCTTTAGATTTGCCA | 1698 | 1717 | 139 |
| 110113 | AATGAAGCAAACTCTATAAA | 1720 | 1739 | 140 |
| 110114 | AGCTTCTTTAGCGTCTTCGA | 1739 | 1758 | 141 |
| 110115 | CCCTTTTATTACAGGAATTT | 1761 | 1780 | 142 |
| 110116 | TGATTGCTCTGCCCTCAATT | 1782 | 1801 | 143 |
| 110117 | TGGGTCCTTGCAACTCCAGC | 1803 | 1822 | 144 |
| 110118 | TCTGGCATTAGGTGATCCCC | 1823 | 1842 | 145 |
| 110119 | ACAGAGTTTTGGATGGCTGG | 1845 | 1864 | 146 |
| 110120 | TCCTCAGACAGGCCTTTGAC | 1867 | 1886 | 147 |
| 110121 | CTTTAATGTCTCTTCAGTGG | 1889 | 1908 | 148 |
| 110122 | GAACGGAGCCGTCAAATGAC | 1911 | 1930 | 149 |
| 110123 | CGGTCAGTAACTATCCTTGC | 1933 | 1952 | 150 |
| 110124 | CAGAAGCTATTCAAACTTCG | 2261 | 2280 | 151 |
| 110125 | TTGATCAGGTAACAGTAAAA | 2326 | 2345 | 152 |
| 110126 | ATACTGTCTTGGAATGTCCT | 2361 | 2380 | 153 |
| 110127 | GATTTCCAAGGAGACCACAG | 2387 | 2406 | 154 |
| 110128 | ACACGGTATTGCCCTTGAAA | 2420 | 2439 | 155 |

HeLa cells were transfected with 15 nM of an antisense oligonucleotide followed by treatment with CHX as described above. RT-qPCR was used to determine antisense activity of each oligonucleotide in ethanol treated cells compared to translation-inhibited CHX treated cells, using the primer probe sets described above.

TABLE 24
NCL1 mRNA levels in HeLa
cells (% control in absence of ASO)

| Compound | | |
|---|---|---|
| No. | Ethanol | CHX |
| 110049 | 68 | 105 |
| 110050 | 93 | 132 |
| 110051 | 79 | 99 |
| 110052 | 61 | 69 |
| 110053 | 69 | 82 |
| 110054 | 66 | 70 |
| 110055 | 41 | 14 |
| 110056 | 66 | 53 |
| 110057 | 75 | 99 |
| 110058 | 47 | 37 |
| 110059 | 73 | 86 |
| 110060 | 58 | 55 |
| 110061 | 57 | 44 |
| 110062 | 32 | 19 |
| 110063 | 42 | 24 |
| 110064 | 53 | 44 |
| 110065 | 87 | 82 |
| 110066 | 80 | 87 |
| 110067 | 68 | 58 |
| 110068 | 36 | 36 |
| 110069 | 33 | 20 |
| 110070 | 28 | 32 |
| 110071 | 36 | 31 |
| 110072 | 95 | 111 |
| 110073 | 35 | 25 |
| 110074 | 15 | 14 |
| 110075 | 28 | 35 |
| 110076 | 37 | 49 |
| 110077 | 28 | 28 |
| 110078 | 26 | 39 |
| 110079 | 48 | 44 |
| 110080 | 17 | 16 |
| 110081 | 42 | 32 |
| 110082 | 29 | 27 |
| 110083 | 43 | 45 |
| 110084 | 32 | 46 |
| 110085 | 39 | 49 |
| 110086 | 39 | 54 |
| 110087 | 48 | 56 |
| 110088 | 65 | 92 |
| 110089 | 48 | 54 |
| 110090 | 62 | 70 |
| 110091 | 40 | 46 |
| 110092 | 70 | 58 |
| 110093 | 44 | 35 |
| 110094 | 60 | 58 |
| 110095 | 51 | 43 |
| 110096 | 68 | 69 |
| 110097 | 54 | 42 |
| 110098 | 45 | 29 |
| 110099 | 44 | 35 |
| 110100 | 57 | 51 |
| 110101 | 52 | 39 |
| 110102 | 34 | 25 |
| 110103 | 68 | 51 |
| 110104 | 80 | 80 |
| 110105 | 54 | 41 |
| 110106 | 46 | 32 |
| 110107 | 49 | 50 |
| 110108 | 63 | 55 |
| 110109 | 48 | 29 |
| 110110 | 56 | 55 |
| 110111 | 58 | 51 |
| 110112 | 50 | 45 |
| 110113 | 82 | 86 |
| 110114 | 46 | 26 |
| 110115 | 42 | 32 |
| 110116 | 62 | 50 |
| 110117 | 60 | 37 |
| 110118 | 58 | 41 |
| 110119 | 58 | 55 |
| 110120 | 58 | 52 |
| 110121 | 51 | 49 |
| 110122 | 84 | 79 |
| 110123 | 44 | 37 |
| 110124 | 87 | 87 |
| 110125 | 80 | 86 |
| 110126 | 37 | 50 |
| 110127 | 47 | 58 |
| 110128 | 45 | 51 |

### Example 7: Accessibility of Specific Portions of mRNA During Translation

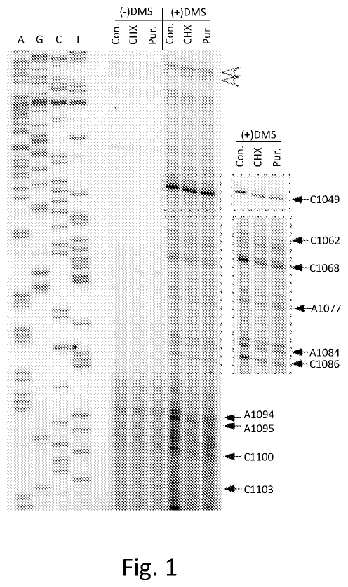

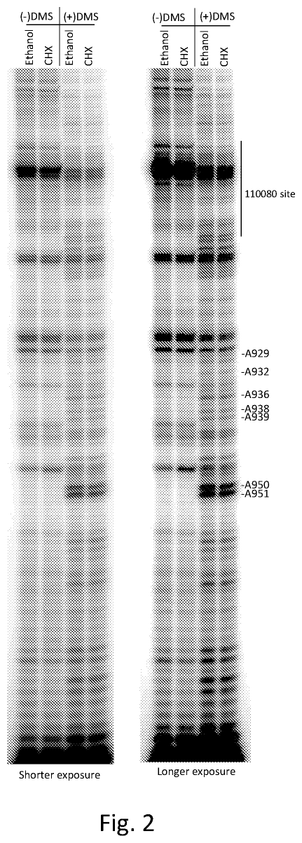

The accessibility of specific portions of mRNAs during translation were assessed via in vivo chemical modification using dimethyl sulfate (DMS), which methylates accessible A and C nucleotides and causes primer extension to terminate one nucleotide prior to these modified nucleotides. This method is described in further detail in Liang, et al, Molecular Cell, 28, 965-977 (2007). In brief, HeLa cells at ˜80% confluence were treated with ethanol (control), 100 μg/mL CHX for 1.5 hours, or 20 μg/mL puromycin for 1.5 hours followed by 100 μg/mL CHX for 15 minutes. Cells were then treated with DMS, and total RNA was harvested. RNA was also harvested for control cells not treated with DMS. Primer extension was performed with 8 μg of total RNA and primer XL845 or primer XL877. Primer XL845 has the sequence TGGCCATTTCCTTCTTTCGTT (SEQ ID NO: 47) and primer XL877 has the sequence AAAACATCGCTGATACCAGT (SEQ ID NO: 48) and was used for both DNA sequencing and primer extension. The primer extension products were analyzed on an 8%, 7M urea PAGE gel and the results were visualized by autoradiography. In the presence of DMS and ethanol or CHX, primer extension signals were approximately the same intensity at the 110080 site and at A929, A932, A936, A938, A939, A950, and A951, indicating that CHX treatment did not change the accessibility of these sites. (See FIG. 1.) In the presence of DMS, primer extension signals were weaker for CHX and puromycin treated samples at C1049, C1062, C1068, A1077, A1084, C1086, A1094, A1095, C1100, and C1103, indicating accessibility of these sites was reduced in the presence of CHX or puromycin. See FIG. 2. These sites overlap with the portions of the target mRNA complementary to antisense oligonucleotide compound nos. 110088, 110089, and 110090.

### Example 8: Effects of Translation Inhibition on Antisense Activities of ASOs and siRNA

Reduction of mRNA with siRNA in the presence of CHX was tested. The siRNAs (“siRNA-110074”, “siRNA-110086”, and “siRNA-110091”) are complementary to the same portions of the target mRNA as antisense oligonucleotide compound numbers 110074, 110086, and 110091, respectively.

HeLa cells were transfected with an antisense oligonucleotide or siRNA followed by treatment with CHX as described above. RT-qPCR was used to determine antisense activities in ethanol treated cells compared to translation-inhibited CHX or puromycin treated cells. The results show that inhibition of translation increased activities of antisense oligonucleotides complementary to accessible portions of NCL1 mRNA but did not increase activities of antisense oligonucleotides complementary to less accessible portions (110086, 110091) or the 3′ UTR (110126, 110128). Activities of siRNAs targeting the same sites as compound nos. 110086 or 110091 were reduced, and activity of an siRNA targeting the same site as compound no. 110074 was not affected. These results show that activity of siRNA was not increased when translation was inhibited regardless of accessibility of the target site.

TABLE 25a
NCL1 mRNA levels in HeLa cells
with and without CHX treatment

| ASO | | [ASO] (nM) | | | | | |
|---|---|---|---|---|---|---|---|
| compound | | 0 | 0.7 | 2.2 | 6.7 | 20 | 60 |
| No. | Cell treatment | NCL1 mRNA (% control) | | | | | |
| 110055 | Ethanol | 100 | 95 | 81 | 65 | 40 | 13 |
| | CHX (100 μg/mL in | 100 | 87 | 66 | 42 | 19 | 6 |
| | EtOH) | | | | | | |
| 110074 | Ethanol | 100 | 95 | 81 | 61 | 31 | 16 |
| | CHX (100 μg/mL in | 100 | 85 | 65 | 46 | 21 | 6 |
| | EtOH) | | | | | | |
| 110080 | Ethanol | 100 | 80 | 62 | 32 | 14 | 6 |
| | CHX (100 μg/mL in | 100 | 45 | 29 | 18 | 10 | 7 |
| | EtOH) | | | | | | |
| 110086 | Ethanol | 100 | 93 | 85 | 66 | 55 | 28 |
| | CHX (100 μg/mL in | 100 | 112 | 93 | 94 | 67 | 56 |
| | EtOH) | | | | | | |
| 110091 | Ethanol | 100 | 87 | 75 | 36 | 13 | 10 |
| | CHX (100 μg/mL in | 100 | 97 | 88 | 56 | 24 | 17 |
| | EtOH) | | | | | | |
| 110126 | Ethanol | 100 | 96 | 85 | 69 | 29 | 18 |
| | CHX (100 μg/mL in | 100 | 98 | 99 | 88 | 49 | 29 |
| | EtOH) | | | | | | |
| 110128 | Ethanol | 100 | 95 | 97 | 72 | 45 | 27 |
| | CHX (100 μg/mL in | 100 | 108 | 106 | 103 | 55 | 31 |
| | EtOH) | | | | | | |

TABLE 25b
NCL1 mRNA levels in HeLa cells
with and without CHX treatment

| | | [siRNA] (nM) | | | | | |
|---|---|---|---|---|---|---|---|
| siRNA | | 0 | 0.0064 | 0.032 | 0.16 | 0.8 | 4 |
| compound | Cell treatment | NCL1 mRNA (% control) | | | | | |
| siRNA- | Ethanol | 100 | 83 | 60 | 33 | 16 | 15 |
| 110074 | CHX (100 μg/mL in | 100 | 85 | 57 | 31 | 18 | 17 |
| | EtOH) | | | | | | |

TABLE 25c
NCL1 mRNA levels in HeLa cells
with and without CHX treatment

| | | [siRNA] (nM) | | | | | |
|---|---|---|---|---|---|---|---|
| siRNA | | 0 | 0.005 | 0.024 | 0.12 | 0.6 | 3 |
| compound | Cell treatment | NCL1 mRNA (% control) | | | | | |
| siRNA- | Ethanol | 100 | 91 | 75 | 68 | 53 | 28 |
| 110086 | CHX (100 μg/mL in | 100 | 103 | 101 | 84 | 66 | 35 |
| | EtOH) | | | | | | |

TABLE 26a
Expression of NCL1 mRNA in HeLa cells
with and without puromycin treatment

| ASO | | | | | | | |
|---|---|---|---|---|---|---|---|
| compound | [ASO] (nM) | 0 | 3.75 | 7.5 | 15 | 30 | 60 |
| no. | Cell treatment | NCL1 mRNA (% control) | | | | | |
| 110091 | Water | 100 | 99 | 90 | 45 | 21 | 17 |
| | Puromycin | 100 | 102 | 98 | 57 | 31 | 27 |

TABLE 26b
Expression of NCL1 mRNA in HeLa cells
with and without puromycin treatment

| | | [siRNA] (nM) | | | | | |
|---|---|---|---|---|---|---|---|
| siRNA | | 0 | 3.75 | 7.5 | 15 | 30 | 60 |
| compound | Cell treatment | NCL1 mRNA (% control) | | | | | |
| siRNA- | Water | 100 | 81 | 82 | 71 | 61 | 43 |
| 110091 | Puromycin | 100 | 104 | 96 | 90 | 62 | 39 |

1. A method comprising
contacting a cell with an antisense compound comprising an antisense oligonucleotide, wherein the nucleobase sequence of the antisense oligonucleotide is complementary to a target mRNA and
contacting the cell with an inhibitor of translation.

2. The method of claim 1, wherein the expression of the target mRNA is reduced.

3. The method of claim 1 or 2, wherein the amount of the target mRNA is reduced.

4. The method of claim 3, wherein the amount of the target mRNA is reduced to a greater extent than the amount of target mRNA reduction that occurs in the absence of the inhibitor of translation.

5. The method of any of claims 1-4, wherein the target mRNA is efficiently translated in the absence of the inhibitor of translation.

6. The method of any of claims 1-5, wherein the target mRNA is enriched in polysomes in the absence of the inhibitor of translation.

7. The method of any of claims 1-6, wherein the target mRNA is enriched in heavy polysomes in the absence of the inhibitor of translation.

8. The method of any of claim 1-7, wherein the target mRNA is not IL-4 receptor, IL-13 receptor, a subunit of an IL-4 receptor, or a subunit of an IL-13 receptor.

9. The method of any of claims 1-8, wherein the nucleobase sequence of the antisense oligonucleotide is complementary to the coding region of the target mRNA.

10. The method of any of claims 1-9, wherein the nucleobase sequence of the antisense oligonucleotide is complementary to a portion of the target mRNA that is accessible during translation.

11. The method of any of claims 1-10, wherein the nucleobase sequence of the antisense oligonucleotide is at least 80% complementary to the target mRNA.

12. The method of any of claims 1-10, wherein the nucleobase sequence of the antisense oligonucleotide is at least 85% complementary to the target mRNA.

13. The method of any of claims 1-10, wherein the nucleobase sequence of the antisense oligonucleotide is at least 90% complementary to the target mRNA.

14. The method of any of claims 1-10, wherein the nucleobase sequence of the antisense oligonucleotide is at least 95% complementary to the target mRNA.

15. The method of any of claims 1-10, wherein the nucleobase sequence of the antisense oligonucleotide is 100% complementary to the target mRNA.

16. The method of any of claims 1-15, wherein the antisense oligonucleotide is a modified oligonucleotide.

17. The method of claim 16, wherein the modified oligonucleotide is a gapmer.

18. The method of claims 16 or 17, wherein the antisense oligonucleotide comprises at least one modified internucleoside linkage.

19. The method of claim 18, wherein the at least one modified internucleoside linkage is a phosphorothioate internucleoside linkage.

20. The method of claim 18, wherein all of the internucleoside linkages of the antisense oligonucleotide are modified internucleoside linkages.

21. The method of claim 20, wherein all of the internucleoside linkages of the antisense oligonucleotide are phosphorothiate internucleoside linkages.

22. The method of claim 19, wherein all of the internucleoside linkages of the antisense oligonucleotide are selected from phosphorothioate and phosphate internucleoside linkages.

23. The method of any of claims 1-22, wherein the antisense compound is single-stranded.

24. The method of claim 23, wherein the antisense compound consists of a conjugate group and the antisense oligonucleotide.

25. The method of claim 24, wherein the antisense compound consists of the antisense oligonucleotide.

26. The method of any of claims 1-25, wherein the inhibitor of translation inhibits translation intiation.

27. The method of any of claims 1-25, wherein the inhibitor of translation inhibits translation elongation.

28. The method of any of claims 1-25, wherein the inhibitor of translation is a second antisense compound comprising a second antisense oligonucleotide.

29. The method of claim 28, wherein the nucleobase sequence of the second antisense oligonucleotide is complementary to the 5′-UTR of the target mRNA.

30. The method of claim 28 or 29, wherein the second antisense oligonucleotide is a modified oligonucleotide that is not a gapmer.

31. The method of claim 30, wherein the second antisense oligonucleotide is a fully modified oligonucleotide.

32. The method of any of claims 1-27, wherein the inhibitor of translation is a small molecule.

33. The method of any of claims 1-27 or 32, wherein the inhibitor of translation is Rapamycin, Everolimus, Temsirolimus, Ridaforolimus, Hippuristanol, or Homoharringtonine.

34. The method of any of claims 1-27, 32 or 33, wherein the inhibitor of translation is puromycin.

35. The method of any of claims 1-27, 32, or 33, wherein the inhibitor of translation is cycloheximide.

36. The method of any of claims 1-27, 32, or 33, wherein the inhibitor of translation is 4E1Rcat.

37. The method of any of claims 1-27, 32, or 33, wherein the inhibitor of translation is lactimidomycin.

38. The method of any of claims 1-37, wherein the inhibitor of translation inhibits eukaryotic translation.

39. The method of any of claims 1-38, wherein the cell is in a population of rapidly proliferating cells.

40. The method of any of claims 1-39, wherein the cell is a tumor cell.

41. The method of any of claims 1-40, wherein the cell is in an animal.

42. The method of claim 41, wherein the animal is a human individual.

43. The method of claim 42 comprising administering the antisense compound and the inhibitor of translation to the individual.

44. The method of claim 43, wherein the individual has a disease or condition that is ameliorated or treated by the administration of the antisense compound.

45. The method of claim 44, wherein the disease or condition is cancer.

46. The method of any of claims 43-45, wherein the antisense compound and the inhibitor of translation are administered simultaneously.

47. The method of any of claims 43-45, wherein the antisense compound and the inhibitor of translation are administered sequentially.

48. Use of an antisense oligonucleotide with a nucleobase sequence complementary to the coding region of a target mRNA in combination with an inhibitor of translation for treatment of a disease.
